(19) United States
(12) Patent Application Publication
(10) Pub. No.: US 2025/0121067 A1
(43) Pub. Date: Apr. 17, 2025

(54) WATER-SOLUBLE CANNABINOID PRODRUGS COMPOSITIONS AND METHODS OF SYNTHESIZING THE SAME

(71) Applicant: Trait Biosciences, Inc., Los Alamos, NM (US)

(72) Inventors: Se-Ho Kim, Los Alamos, NM (US); Shan Hazoor, Santa Fe, NM (US)

(21) Appl. No.: 18/678,152

(22) Filed: May 30, 2024

Related U.S. Application Data
(63) Continuation-in-part of application No. PCT/US23/23985, filed on May 31, 2023.
(60) Provisional application No. 63/347,247, filed on May 31, 2022.

(51) Int. Cl. A61K 47/54

(52) U.S. Cl. CPC A61K 47/542

(57) ABSTRACT

This invention relates to the compositions and methods of synthesizing water-soluble cannabinoid prodrugs that are conjugated with amino acids, amino sugars, and aminosulfonic acid derivatives through a carbamate linker. The invention also includes various salt formations of the conjugates that contain an acid group. Although this invention can be applied to a range of cannabinoids, compounds such as CBD, THC, CBN, and CBG are especially useful due to their potential use in the food and beverage, and pharmaceutical industries.

### CROSS-REFERENCE TO RELATED APPLICATIONS

This U.S. bypass continuation-in-part claims the benefit of and priority to International PCT application PCT/US2023/023985, filed May 31, 2023, which claims the benefit of and priority to U.S. Provisional Application No. 63/347,247, filed May 31, 2022, the specification, claims and drawings of which are incorporated herein by reference in their entirety.

### TECHNICAL FIELD

The present invention is directed to novel chemical compositions of matter, and in particular novel cannabinoid prodrug compounds, and in particular novel cannabinoids prodrugs conjugated with amino acids, amino sugars, and aminosulfonic acid derivatives, and pharmaceutical acceptable salts of the same.

### BACKGROUND

With the changes of regulatory environment and consumer perception, hemp- and marijuana-derived cannabinoids, such as cannabidiol (CBD) and tetrahydrocannabinol (THC) are widely used in consumer products such as food, beverages, supplements, personal care products, and cosmetics. In just one example, the global market value of CBD-containing products is projected to grow from $591 million in 2018 to $22 billion in 2022. In particular, edible cannabinoids in food and recreational beverage products are an increasingly popular route of cannabinoid consumption and has become a fast-growing subsector in the industry. However, when orally administered, edible cannabinoid products not only delays reaching a proper concentration in blood and tissue, but also leads to a low and variable oral bioavailability compared to inhalation (smoking). Several factors could account for underlying mechanisms of the undesired pharmacokinetic behaviors, including poor solubility of cannabinoids in aqueous environment, incomplete gastrointestinal absorption, instability in gastric pH, extensive hepatic metabolism, and drug-drug interactions. For example, the high hydrophobicity (log P 5.91) and intrinsically low solubility of CBD (0.1 μg/mL) are considered as the major contributing factor to its unpredictable oral bioavailability (International Journal of Pharmaceutics 2020, 589, 119812).

In this context, large investments in research have been dedicated to enhancing water solubility of cannabinoids, such as CBD among others, through several techniques that are designed to modulate physicochemical properties affecting water solubility (e.g., surface charge, particle size, shape, physical form, molecular symmetry, chemical structure, pH, emulsifier, and stabilizer). More specifically, these technologies include formulation of solid dispersion and lipid nanoparticles, solubilization in protein or lipid-based carrier system, the use of alternative solid state (polymorphs or cocrystals), covalent chemical modification (generation of prodrugs), and salt formation.

Among these, new nano-based cannabinoid delivery systems and several synthetic cannabinoids analogs have provided promising results such as enhanced bioavailability, reduced clearance, enhanced target-specific delivery, and improved pharmacological potency in preclinical and clinical studies. However, the toxicity and regulatory concerns need to be considered for their applications in food and beverages products. As such, there exists a long-felt need for a safe and cost-effective system to enhance cannabinoid solubility and enable delivery in aqueous solutions and formulations.

### SUMMARY OF THE INVENTION

One aspect of the invention may include novel conjugated cannabinoid prodrug compounds, and their methods of synthesis. In one preferred aspect, the invention includes a cannabinoid having at least one conjugation site that may be coupled with a promoiety through a linker. In this aspect, the cannabinoid prodrug of the invention may be conjugated with one or more promoiety, such as an amino acid, amino sugar, or aminosulfonic acid derivative at a conjugation site by a carbamate bond linker.

Another aspect of the current invention includes systems, methods, and compositions for the generation of one or more novel conjugated cannabinoid prodrug compounds, which may include their corresponding salt forms.

In a preferred aspect the invention may include cannabinoid prodrug compounds, or a pharmaceutically acceptable salts thereof, selected from the Group consisting of the compounds of Formulas I-IV. In this embodiment, the cannabinoid prodrug compounds, or a pharmaceutically acceptable salts thereof, may include a cannabinoid prodrug compound, wherein the cannabinoid is selected from the group consisting of: CBD, THC, cannabinol (CBN) cannabigerol (CBG), and their acidic forms, or a combination of the same.

In another aspect, the cannabinoid prodrug compounds, or a pharmaceutically acceptable salts thereof, may include a cannabinoid prodrug compound, wherein the cannabinoid includes at least one conjugation site, such as a hydroxyl (—OH) or carboxyl group (—COOH) group, selected from: delta-Δ⁹-tetrahydrocannabinol (THC), delta-Δ⁸-tetrahydrocannabinol (Delta-8-THC), 11-Hydroxy-Δ⁹-tetrahydrocannabinol (11-OH-THC), tetrahydrocannabinolic acid (THCA), cannabidiol (CBD), cannabichromene (CBC), cannabigerol (CBG), cannabigerolic acid (CBGA), cannabinol (CBN), cannabinolic acid (CBNA), cannabidiolic acid (CBDA), cannabicyclol (CBL), cannabivarin (CBV), tetrahydrocannabivarin (THCV), cannabigerovarin (CBGV), cannabichromevarin (CBCV), cannabidivarin (CBDV), cannabicyclol (CBL), cannabielsoin (CBE), cannabifuran (CBF); and cannabinodiol (CBDN).

Another aspect of the current invention includes pharmaceutical compositions containing one or novel conjugated cannabinoid prodrug compounds, and their use to treat one or more disease conditions in a subject in need thereof. Another aspect of the current invention includes consumer products, such as food and beverage additives, nutraceuticals, topical compositions, all containing one or novel conjugated cannabinoid prodrug compounds of the invention.

Additional aspects of the invention may become evident based on the specification and figures presented below.

### BRIEF DESCRIPTION OF THE DRAWINGS

FIG. 1. Comparison of absorption pathways of CBD and water-soluble carbamate prodrugs of CBD in GI tract.

FIG. 2. Exemplary cannabinoids with specific conjugation site identified.

FIG. 3. Cannabinoid pro-drug synthesis Scheme 1, in one embodiment thereof.

FIG. 4. Cannabinoid pro-drug synthesis Scheme 2, in one embodiment thereof.

FIG. 5. Cannabinoid pro-drug synthesis Scheme 3, in one embodiment thereof.

FIG. 6. Cannabinoid pro-drug synthesis Scheme 4, in one embodiment thereof.

FIG. 7. Cannabinoid pro-drug synthesis Scheme 5, in one embodiment thereof.

FIG. 8. Cannabinoid pro-drug synthesis Scheme 6, in one embodiment thereof.

### DETAILED DESCRIPTION OF THE INVENTION

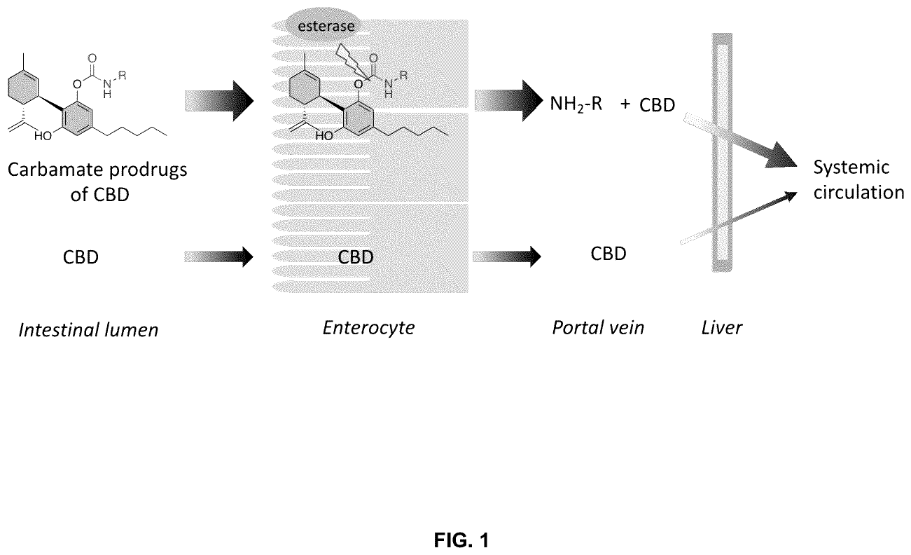

The invention may include novel conjugated cannabinoid prodrug compounds wherein a cannabinoid, having at least one conjugation site is coupled with a promoiety by a linker. As shown in FIG. 1, the present inventors have developed a novel prodrug strategy is to use a carbamate linker with water-soluble moieties classified as GRAS (generally recognized as safe) grade such as amino acids, amino sugars, or aminosulfonic acid derivatives. The carbamate linkages are stable under acidic conditions but can be rapidly cleaved by esterases highly expressed in the small intestine to release the cannabinoid before absorption. Consequently, this approach can prevent precipitation of cannabinoids, such as CBD or THC in the stomach and premature release in the systemic circulation, which increases the prodrugs bioequivalence to un-conjugated cannabinoids.

In one preferred embodiment, a cannabinoid, or a pharmaceutically acceptable salt thereof, having at least one conjugation site coupled with a promoiety through a linker comprising a carbamate bond. As shown below, the exemplary conjugated CBD prodrug compounds of the invention, identified as Formula IA and IIA include one or two conjugation sites that can be coupled with a promoiety through a carbamate bond linker. As described below, the promoiety conjugated with the CBD compounds through a linker may include amino acids, amino sugars, or aminosulfonic acid derivatives.

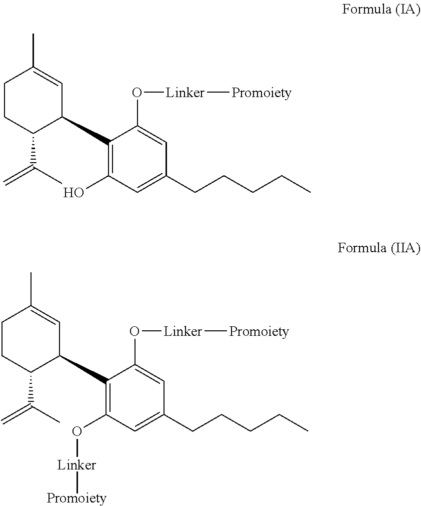

Additional exemplary cannabinoids that may be conjugated with one or more promoiety to form a conjugated cannabinoid prodrug may include one or more of the following:

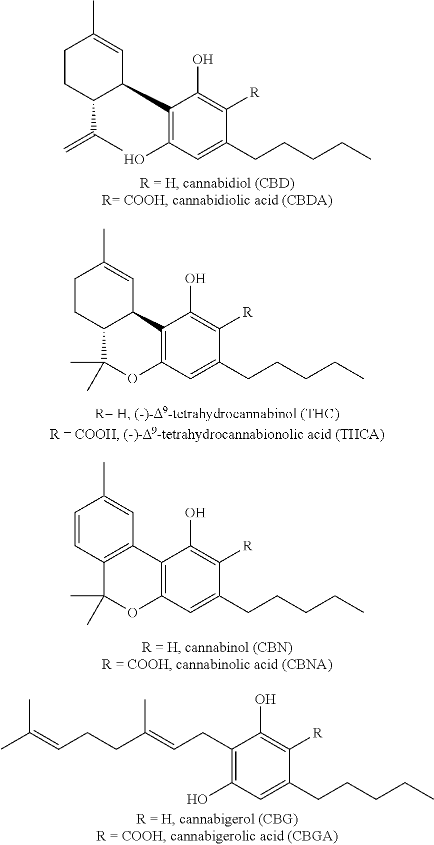

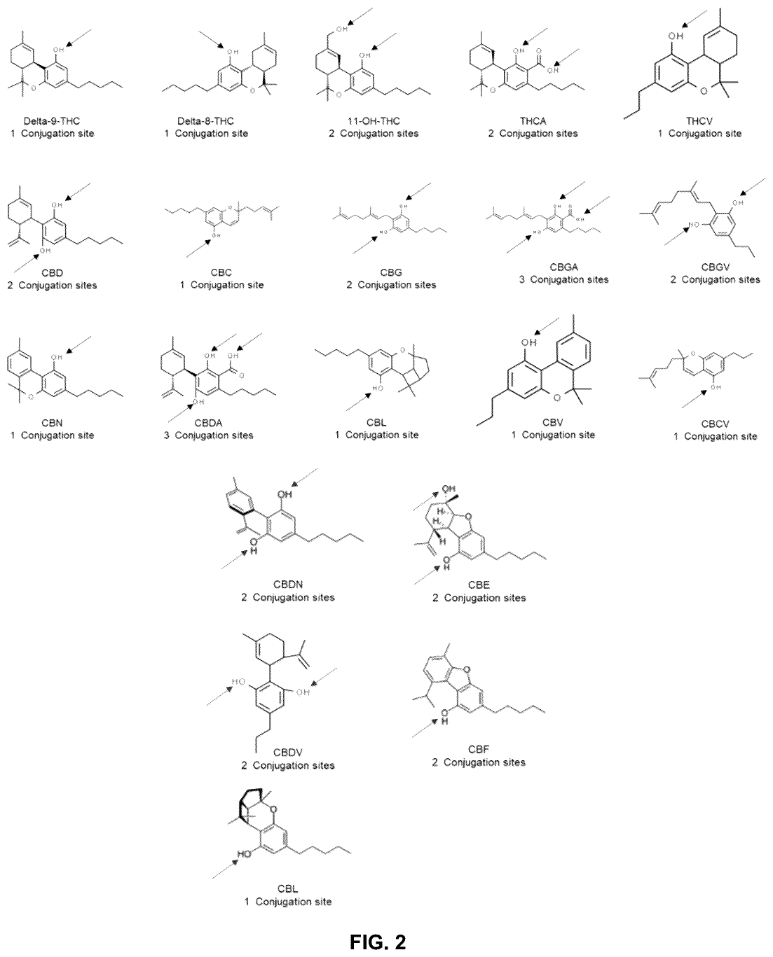

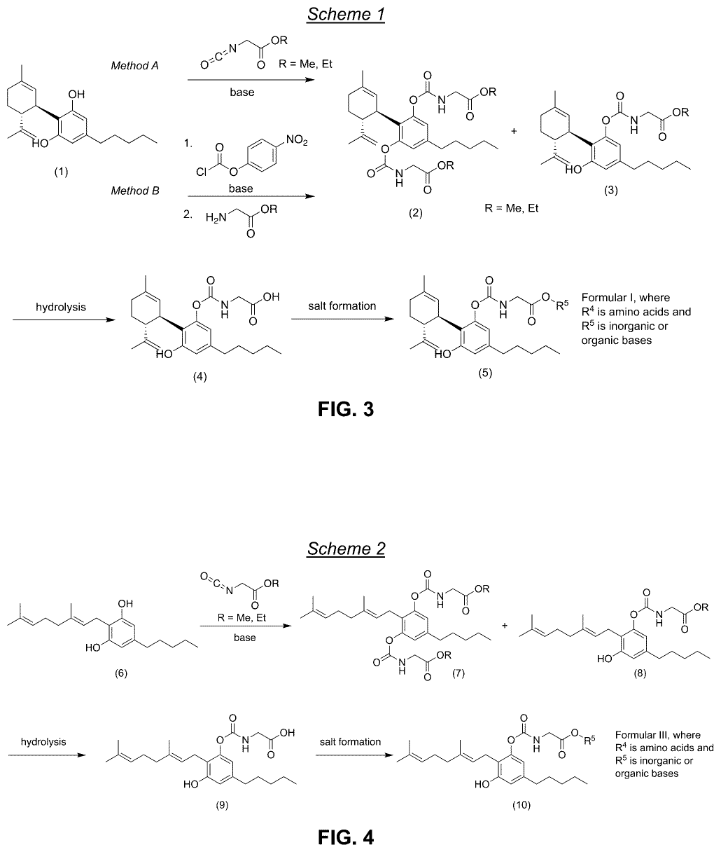

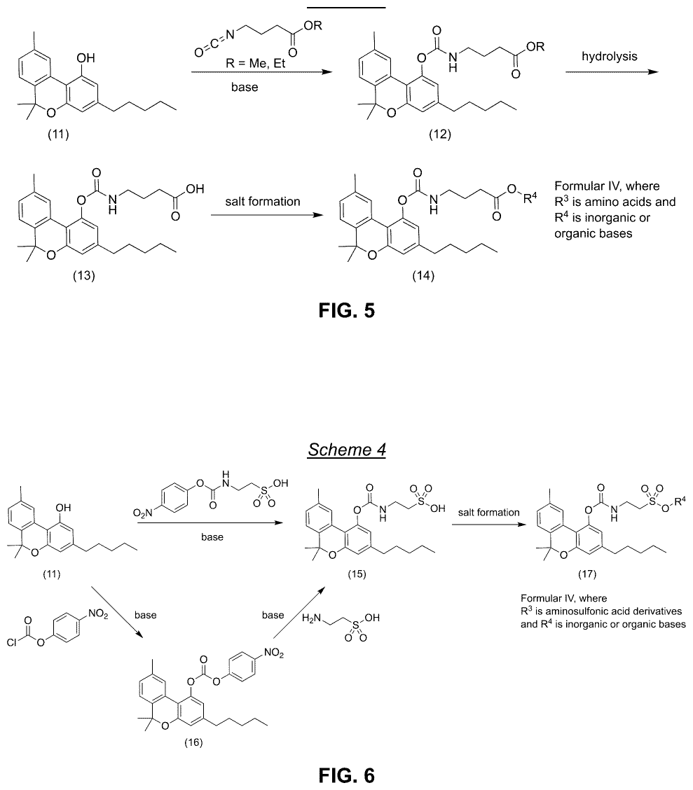

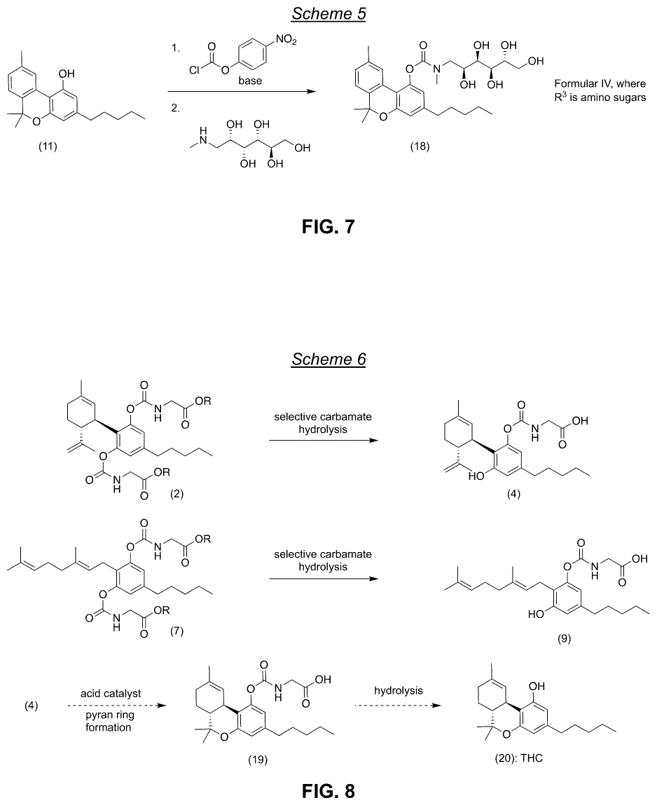

In another preferred embodiment, the invention may include a cannabinoid, or a pharmaceutically acceptable salt thereof, having at least one conjugation site coupled with a promoiety through a linker comprising a carbamate bond. As shown below, a cannabinoid prodrug compound of the invention may include a cannabinoid having at least one conjugation site, such as a hydroxyl group, that may be coupled with a promoiety through a linker comprising a carbamate or an ester bond. Exemplary cannabinoids and their conjugation sites include, but are not limited to the cannabinoids, with specific conjugation site identified in FIG. 2.

As noted above, a promoiety of the invention may be selected from the group consisting of an amino acid, an amino sugar, and aminosulfonic acid derivative, or a combination of the same. The scope, as well as the chemical structures/characteristics of the promoieties of the invention are known and readily understood by those of ordinary skill in the art.

In a preferred embodiment, an amino acid promoiety of the invention can include, but not limited to: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and GABA (gamma-aminobutyric acid), among others.

In a preferred embodiment, the amino sugar promoiety of the invention can include: meglumine, glucosamine, galactosamine, sialic acid, and Daunosamine, Mannosamine, Allosamine, Altrosamine, Idosamine, Talosamine, N-Acetyl-D-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, N-Acetyl-6-deoxy-L-altrosamine, N-Acetyl-6-deoxy-D-talosamine, among others.

In a preferred embodiment, the aminosulfonic acid promoiety of the invention can include: taurine and taurine derivatives including homotaurine and cysteic acid, among others.

In a preferred embodiment, the invention includes a cannabinoid prodrug compound according to Formula I, comprising:

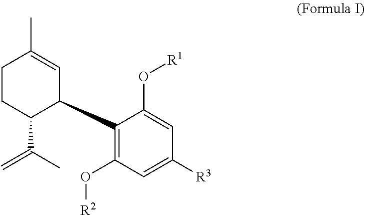

wherein,
R¹ is H, —C(═O)R⁴;
R² is H, —C(═O)R⁴;
R³ is H or linear alkane;
R⁴ is amino acid, amino sugar, or aminosulfonic acid derivative.
   wherein at least one of R¹ or R² is C(═O)R⁴
   or a pharmaceutically acceptable salt thereof.

In another preferred embodiment, the invention includes a cannabinoid prodrug compound according to Formula I, comprising:

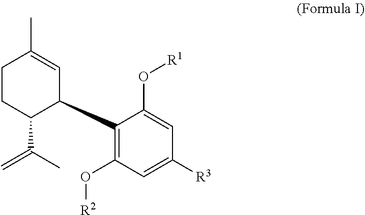

wherein,
R³ is C₅ linear alkane;
R⁴ is an amino acid-(R⁵), amino acid sugar or aminosulfonic acid derivative-(R⁵); and
R⁵ is H, Na, K, Ca, Mg, lysine, arginine, histidine, amino sugar, diethylaminoethanol, or tris base.

In a preferred embodiment, the invention includes a cannabinoid prodrug compound according to Formula II, comprising:

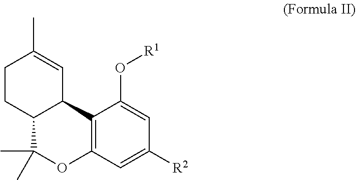

wherein,
R¹ is C(═O)(R³);
R² is H or linear alkane;
R³ is amino acid, amino sugar, or aminosulfonic acid derivative;
   or a pharmaceutically acceptable salt thereof.

In another preferred embodiment, the invention includes a cannabinoid prodrug compound according to Formula II, comprising:

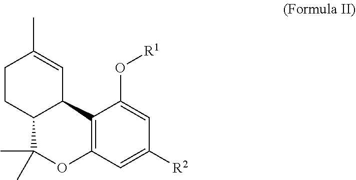

wherein,
R² is C₅ linear alkane;
R³ is an amino acid-(R⁴), amino acid sugar, or aminosulfonic acid derivative-(R⁴); and
R⁴ is H, Na, K, Ca, Mg, lysine, arginine, histidine, amino sugar, diethylaminoethanol, or tris base;
or a pharmaceutically acceptable salt thereof.

In another preferred embodiment, the invention includes a cannabinoid prodrug compound according to Formula III, comprising:

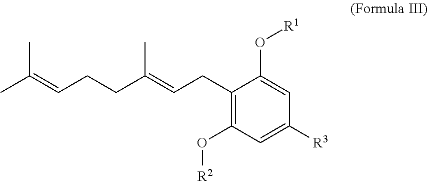

wherein,
R¹ is H, —C(═O)(R⁴);
R² is H, —C(═O)(R⁴);
R³ is H or linear alkane;
R⁴ is amino acid, amino acid sugar, or aminosulfonic acid derivative;
   wherein at least one of R¹ or R² is —C(═O)(R⁴);
   or a pharmaceutically acceptable salt thereof.

In another preferred embodiment, the invention includes a cannabinoid prodrug compound according to Formula III, comprising:

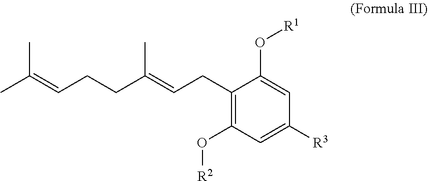

wherein,
R³ is C₅ linear alkane;
R⁴ is an amino acid-(R⁵), amino acid sugar, or aminosulfonic acid derivative-(R⁵); and
R⁵ is H, Na, K, Ca, Mg, lysine, arginine, histidine, amino sugar, diethylaminoethanol, or tris base.
   In a preferred embodiment, the invention includes a cannabinoid prodrug compound according to Formula IV, comprising:

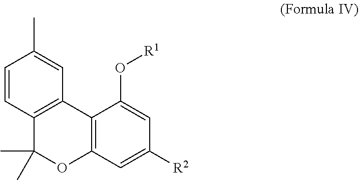

wherein,
R¹ is —C(═O)(R³);
R² is H or linear alkane;
R³ is amino acid, amino sugar, or aminosulfonic acid derivative;
   or a pharmaceutically acceptable salt thereof.

In another preferred embodiment, the invention includes a cannabinoid prodrug compound according to Formula IV, comprising:

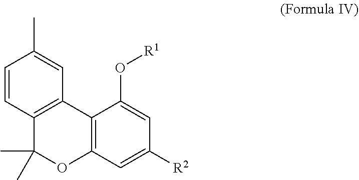

wherein,
R² is C₅ linear alkane;
R³ is an amino acid-(R⁴), amino sugar, or aminosulfonic acid derivative-(R⁴); and
R⁴ is H, Na, K, Ca, Mg, lysine, arginine, histidine, amino sugar, diethylaminoethanol, or tris base.

In preferred embodiments, the amino acid conjugate of the compound of Formula I-IV can be selected from the group consisting of: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and GABA (gamma-aminobutyric acid).

In preferred embodiments, the amino sugar conjugate of the compound of Formula I-IV can be selected from the group consisting of: meglumine, glucosamine, galactosamine, sialic acid, and Daunosamine, Mannosamine, Allosamine, Altrosamine, Idosamine, Talosamine, N-Acetyl-D-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, N-Acetyl-6-deoxy-L-altrosamine, N-Acetyl-6-deoxy-D-talosamine.

In a preferred embodiment, the aminosulfonic acid promoiety of the invention can include: taurine and taurine derivatives including homotaurine and cysteic acid, among others.

Additional embodiments of the invention include a pharmaceutical composition comprising at least one of the compounds of any of Formula I-IV, and a pharmaceutically acceptable carrier. The conjugated cannabinoid prodrug compounds of the invention, preferably in the form of a pharmaceutical compositions may include a method for treating a disease condition, comprising the steps of administering a therapeutically effective amount of the pharmaceutical compositions of Formula I-IV to a subject in need thereof. In one embodiment of the invention, a therapeutically effective amount of one or more novel conjugated cannabinoid prodrugs, may be administered to a subject in need thereof, by a route selected from the group consisting of: transdermal, topical, oral, buccal, sublingual, intra-venous, intra-muscular, vaginal, rectal, ocular, nasal and follicular.

Exemplary, disease conditions that can be treated by a cannabinoid prodrug compounds of the invention may be selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis.

One embodiment of the invention includes compositions of matter containing one or more novel conjugated cannabinoid prodrugs, and preferably consumer products containing one or more of the novel cannabinoids of according to Formulas I-IV.

One embodiment of the invention includes compositions of matter containing one or more novel conjugated cannabinoid prodrugs, and preferably food and drink additives containing one or more of the novel conjugated cannabinoid prodrugs according to Formulas I-IV.

One embodiment of the invention includes compositions of matter containing one or more novel conjugated cannabinoid prodrugs, and preferably topical compositions containing one or more novel conjugated cannabinoid prodrugs according to Formulas I-IV.

One embodiment of the invention includes compositions of matter containing one or more novel conjugated cannabinoid prodrugs, and preferably nutraceutical and OTC medication compositions containing one or more novel conjugated cannabinoid prodrugs according to Formulas I-IV.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

All structures depicted herein, unless otherwise stated include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention. The term “stereoisomer” refers to a molecule that is an enantiomer, diastereomer or geometric isomer of a molecule. Stereoisomers, unlike structural isomers, do not differ with respect to the number and types of atoms in the molecule's structure but with respect to the spatial arrangement of the molecule's atoms. Examples of stereoisomers include the (+) and (−) forms of optically active molecules.

As used herein, the term “cannabinoid” may also include different modified forms of a cannabinoid such as a methylated, acetylated, hydroxylated cannabinoids or cannabinoid carboxylic acids. Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methyl ether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3,4,5,6-tetrahydro-7-hydroxy-alpha-alpha-2-trimethyl-9-n-propyl-2, 6-methano-2H-1-benzoxocin-5-methanol-cannabiripsol, trihydroxy-delta-9-tetrahydrocannabinol, and cannabinol.

A cannabinoid may include one or more conjugate sites or conjugation sites that can bind to a promoiety through a linker. As used herein “conjugate site” or “conjugation site” mean a position on a cannabinoid compound that may covalently bind to promoiety directly, or preferably through a linker that is coupled with promoiety, in one preferred embodiment, a “conjugate site” or “conjugation site” may include an —OH or a —COOH group on a cannabinoid. Exemplary conjugation sites are demonstrated in FIG. 2.

The term “compound,” or “compound of the invention” includes all solvates, complexes, polymorphs, radiolabeled derivatives, tautomers, stereoisomers, and optical isomers of the novel conjugated cannabinoid prodrug compounds generally described herein, and salts thereof, unless otherwise specified. Notably, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as histidine, lysine and arginine. An example of a common quaternary ammonium ion is N(CH₃)₄⁺.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc. It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999). For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases, as an N-oxide (>NO). For example, a carboxylic acid group may be protected as an ester for example, as: a C₁₋₇ alkyl ester (e.g., a methyl ester; a t-butyl ester); a C₁₋₇haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g., a benzyl ester; a nitrobenzyl ester); or as an amide, for example, as a methyl amide.

An “R-group” or “substituent” refers to a single atom (for example, a halogen atom) or a group of two or more atoms that are covalently bonded to each other, which are covalently bonded to an atom or atoms in a molecule to satisfy the valency requirements of the atom or atoms of the molecule, typically in place of a hydrogen atom.

“Carbonate” as used here means a substituent, moiety or group that contains a —O—C(═O)—O— structure (i.e., carbonate functional group). Typically, carbonate groups as used here comprise or consist of an organic moiety, wherein the organic moiety is as described herein for an organic moiety bonded to an ester functional group, bonded through the —O—C(═O)—O— structure, e.g., organic moiety —O—C(═O)—O—. When carbonate is used as a Markush group (i.e., a substituent) one of the singly bonded oxygen atoms of the carbonate functional group is attached to a Markush formula with which it is associated and the other is bonded to a carbon atom of an organic moiety as previously described for an organic moiety bonded to an ester functional group.

“Carbamate” as used here means a substituent, moiety or group that contains a structure represented by —O—C(═O)N(Ra)- (i.e., carbamate functional group) or —O—C(═O)N(Ra)2, —O—C(═O)NH(optionally substituted alkyl) or —O—C(═O)N(optionally substituted alkyl)2 (i.e., exemplary carbamate substituents) wherein Ra and optionally substituted alkyl are independently selected wherein Ra, independently selected, is hydrogen, a protecting group or an organic moiety, wherein the organic moiety is as described herein for an organic moiety bonded to an ester functional group and is typically an optionally substituted alkyl. Typically, carbamate groups as used herein comprise or consist of an organic moiety, independently selected from Ra, wherein the organic moiety is as described herein for an organic moiety bonded to an ester functional group, bonded through the —O—C(═O)—N(Ra)- structure, wherein the resulting structure has the formula of organic moiety —O—C(═O)—N(Ra)- or —O—C(═O)—N(Ra)-organic moiety. When carbamate is used as a Markush group (i.e., a substituent), the singly bonded oxygen (O-linked) or nitrogen (N-linked) of the carbamate functional group is attached to a Markush formula with which it is associated. The linkage of the carbamate substituent is either explicitly stated (N- or O-linked) or implicit in the context to which this substituent is referred.

The term “aminosulfonic acid derivatives” and salts thereof have the following general molecular formula:

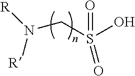

Wherein R and R′ are independently selected from the group consisting of hydrogen, alkyl, cyclohexyl, alkoxy, optionally substituted organic groups having one or more hydroxyl groups, optionally substituted organic amide groups, optionally substituted organic sulfonic acids, optionally substituted organic carboxylic acids, optionally substituted organic carboxylic esters, optionally substituted organic amines, and combinations thereof; n is 1 to 10. Organic aminosulfonic acid derivatives and salts thereof are primary amine-based organic sulfonic acid molecules when R=R′=hydrogen atom, are secondary amine-based organic sulfonic acid molecules when one of R and R′ is hydrogen bonded to a nitrogen atom in the molecule, and are tertiary amine-based organic sulfonic acid molecule when R and R′ are both not hydrogen atoms. Examples of the organic aminosulfonic acid derivatives and salts thereof include sulfonic acid, 2-[(2-hydroxy-1,1-bis(hydroxymethyl)ethyl)amino]ethanesulfonic acid (TES), N-tris(hydroxymethyl)methyl-3-aminopropanesulfonic acid (TAPS), N-tris(hydroxymethyl)methyl-4-aminobutanesulfonic acid (TABS), N-(2-Acetamido)-2-aminoethanesulfonic acid (ACES), N,N-bis(2-hydroxyethyl)-2-aminoethanesulfonic acid (BES), 3-(cyclohexylamino)propane-1-sulfonic acid (CAPS), but are not limited to, taurine, cysteic acid, N-cyclohexyltaurine (CHES), and 2-(((4-nitrophenoxy)carbonyl)amino)ethane-1-sulfonic acid.

The term “linear alkane” is used to refer to an alkane in which each carbon atom is bound to a maximum of two carbon atoms.

The term “ester,” as used herein, alone or in combination, refers to a carboxy group bridging two moieties linked at carbon atoms.

The term “amino” as used herein refers to the group —NH2. The term “alkylamino” refers to amino groups where one or both hydrogen atoms are replaced by a hydrocarbon group He as described above, wherein the amino nitrogen “N” can be substituted by one or two He groups as set forth for alkoxy groups described above. Exemplary alkylamino groups include methylamino, dimethylamino, ethylamino, diethylamino, etc. Also, the term “substituted amino” refers to amino groups where one or both hydrogen atoms are replaced by a hydrocarbon group He as described above, wherein the amino nitrogen “N” can be substituted by one or two He groups as set forth for alkoxy groups described above.

The term “amino acid” generally refers to an organic compound comprising both a carboxylic acid group and an amine group. The term “amino acid” includes both “natural” and “unnatural” or “non-natural” amino acids. Additionally, the term amino acid includes O-alkylated or N-alkylated amino acids, as well as amino acids having nitrogen or oxygen-containing side chains (such as Lys, Orn, or Ser) in which the nitrogen or oxygen atom has been acylated or alkylated. Amino acids may be pure L or D isomers or mixtures of L and D isomers, including racemic mixtures. In a preferred embodiment herein, an amino acid may be conjugated, for example through a carbamate linker to a cannabinoid having a conjugation site.

The term “amino acid sugar,” or “amino sugar,” as used herein refers to monosaccharides having one alcoholic hydroxyl group (commonly but not necessarily in the ‘2-position’) replaced by an amino group, systematically known as x-deoxy-x-monosaccharides. By way of non-limiting example, D-glucosamine or 2-amino-2-deoxy-D-glucopyranose is an amino sugar. Other illustrative amino sugars include but are not limited to erythrosamine, threosamine, ribosamine, arabinosamine, xylosamine, lyxosamine, allosamine, altrosamine, glucosamine, mannosamine, idosamine, galactosamine, talosamine, and their derivatives, all of which are suitable for use within the compositions of the present disclosure. The amino sugars include both aldose and ketose sugars. Additionally, the amino sugars may be of a straight-chain structure; however, the aldehyde or ketone group of the amino sugar may react with a hydroxyl group on a different carbon atom to form a hemiacetal or hemiketal, in which case there is an oxygen bridge between the two carbon atoms, forming a heterocyclic ring. Amino sugar rings with five and six atoms are called furanose and pyranose forms, respectively and exist in equilibrium with their corresponding straight-chain form. It should be noted that the ring form has one more optically active carbon than the straight-chain form, and so has both an a- and a p-form, which interconvert in equilibrium. The term “amino sugar” also means glycosylamines, amino sugars where the nitrogen is substituted with a functional group other than H. Illustrative, non-limiting examples of glycosylamines include N-acetylglucosamine (NAG) and N-methylglucosamine. In a preferred embodiment herein, an amino acid may be conjugated, for example through a carbamate linker to a cannabinoid having a conjugation site.

The term “linker,” as used herein described a chemical bond between a cannabinoid and a promoiety. In a preferred embodiment a liner of the invention includes a carbamate bond or an ester bond. The term “promoiety,” refers to a portion of a prodrug that is not a drug. In a preferred embodiment, a promoiety includes an amino acid, an amino acid sugar, a sweetener, or a depsipeptide that may be conjugated to a cannabinoid by a linker, such as a carbamate bond or an ester bond.

In certain embodiments, it may be convenient or desirable to prepare, purify, and/or handle the active compound in the form of a prodrug. The term “prodrug,” as used herein, pertains to a compound which, when metabolized (e.g., in vivo), yields the desired active compound. Typically, the prodrug is inactive, or less active than the active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include, but are not limited to, those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleaved by phosphatase enzymes during metabolism to yield the active drug with the hydroxy group. Further “prodrugs” include carbamates and carbonates as described herein. Also, some prodrugs are activated enzymatically to yield the active compound, or a compound which, upon further chemical reaction, yields the active compound. For example, the prodrug may be a sugar derivative or other glycoside conjugate or may be an amino acid ester derivative. “Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical Formulation techniques such as those disclosed in Remington's Pharmaceutical Sciences, Mack Publishing Co, Easton, Pa. (19th Edition). The terms “pharmaceutically acceptable salt” refers to salts or esters prepared by conventional means that include salts, e.g, of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned above are meant to comprise the therapeutically active non-toxic acid and base addition salt forms which the compounds can form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic, and like acids. Conversely, these salt forms can be converted into the free base form by treatment with an appropriate base.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine, and the like.

Some of the compounds described herein may also exist in their tautomeric form.

The terms “approximately” and “about” refer to a quantity, level, value, or amount that varies by as much as 30%, or in another embodiment by as much as 20%, and in a third embodiment by as much as 10% to a reference quantity, level, value, or amount. As used herein, the singular form “a,” “an,” and “the” include plural references unless the context clearly dictates otherwise.

The inventive technology may further include novel water-soluble conjugated cannabinoids, and preferably the cannabinoid conjugate prodrugs of the invention. In one preferred embodiment, the invention may include a pharmaceutical composition as an active ingredient an effective amount or dose of one or more of the novel cannabinoid conjugate prodrugs of the invention. In some instances, the active ingredient may be provided together with pharmaceutically tolerable adjuvants and/or excipients in the pharmaceutical composition. Such pharmaceutical composition may optionally be in combination with one or more further active ingredients. In one embodiment, one of the aforementioned the novel cannabinoid conjugate prodrugs of the invention, whereby a promoiety may be removed after administration and/or uptake of a therapeutically effective amount, or effective dose, or dose.

The terms “therapeutically effective amount” or “effective dose” or “dose” are interchangeably used herein and denote an amount of the pharmaceutical compound having a prophylactically or therapeutically relevant effect on a disease or pathological conditions, i.e. which causes in a tissue, system, animal or human a biological or medical response which is sought or desired, for example, by a researcher or physician. Pharmaceutical Formulations can be administered in the form of dosage units which comprise a predetermined amount of active ingredient per dosage unit. The concentration of the prophylactically or therapeutically active ingredient in the Formulation may vary from about 0.1 to 100 wt %. Preferably, a cannabinoid conjugate prodrug of the invention or the pharmaceutically acceptable salts thereof are administered in doses of approximately 0.5 to 1000 mg, more preferably between 0.1 mg and 1000 mg, 1 and 700 mg, and most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors as already described in the present specification (e.g. depending on the condition treated, the method of administration and the age, weight, and condition of the patient). Preferred dosage unit Formulations are those which comprise a daily dose or part-dose, as indicated above, or a corresponding fraction thereof of an active ingredient. Furthermore, pharmaceutical Formulations of this type can be prepared using a process which is generally known in the pharmaceutical art.

As used herein, a consumer product, including a food additive, a beverage additive as well as nutraceutical compositions are described by Sayre et al., in U.S. application Ser. No. 16/110,954. The descriptions of the compositions in paragraphs 0206 to 0251, and the section entitled Preserved Clauses at paragraphs 0358 to 0427, being specifically incorporated hereby reference incorporate by reference.

In one embodiment, the invention may include one or more methods of treating a medical condition in a mammal. In this embodiment, the novel method may include of administering a therapeutically effective amount of a conjugated cannabinoid, for example, at least one conjugated cannabinoid prodrug, wherein the medical condition is selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g, Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis. In a preferred embodiment, the pharmaceutical composition may be administered by a route selected from the group consisting of transdermal, topical, oral, buccal, sublingual, intra-venous, intra-muscular, vaginal, rectal, ocular, nasal, and follicular. The amount of conjugated cannabinoids may be a therapeutically effective amount, which may be determined by the patient's age, weight, medical condition cannabinoid-delivered, route of delivery, and the like. In one embodiment, a therapeutically effective amount may be 50 mg or less of a conjugated cannabinoid. In another embodiment, a therapeutically effective amount may be 50 mg or more of a conjugated cannabinoid.

It should be noted that for any of the above composition, unless otherwise stated, an effective amount of conjugated cannabinoids may include amounts between: 0.01 mg to 0.1 mg; 0.01 mg to 0.5 mg; 0.01 mg to 1 mg; 0.01 mg to 5 mg; 0.01 mg to 10 mg; 0.01 mg to 25 mg; 0.01 mg to 50 mg; 0.01 mg to 75 mg; 0.01 mg to 100 mg; 0.01 mg to 125 mg; 0.01 mg to 150 mg; 0.01 mg to 175 mg; 0.01 mg to 200 mg; 0.01 mg to 225 mg; 0.01 mg to 250 mg; 0.01 mg to 275 mg; 0.01 mg to 300 mg; 0.01 mg 20 to 225 mg; 0.01 mg to 350 mg; 0.01 mg to 375 mg; 0.01 mg to 400 mg; 0.01 mg to 425 mg; 0.01 mg to 450 mg; 0.01 mg to 475 mg; 0.01 mg to 500 mg; 0.01 mg to 525 mg; 0.01 mg to 550 mg; 0.01 mg to 575 mg; 0.01 mg to 600 mg; 0.01 mg to 625 mg; 0.01 mg to 650 mg; 0.01 mg to 675 mg; 0.01 mg to 700 mg; 0.01 mg to 725 mg; 0.01 mg to 750 mg; 0.01 mg to 775 mg; 0.01 mg to 800 mg; 0.01 mg to 825 mg; 0.01 mg to 950 mg; 0.01 mg to 875 mg; 0.01 mg to 900 mg; 0.01 mg to 925 mg; 0.01 mg to 950 mg; 0.01 mg to 975 mg; 0.01 mg to 1000 mg; 0.01 mg to 2000 mg; 0.01 mg to 3000 mg; 0.01 mg to 4000 mg; 01 mg to 5000 mg; 0.01 mg to 0.1 mg/kg.; 0.01 mg to 0.5 mg/kg; 01 mg to 1 mg/kg; 0.01 mg to 5 mg/kg; 0.01 mg to 10 mg/kg; 0.01 mg to 25 mg/kg; 0.01 mg to 50 mg/kg; 0.01 mg to 75 mg/kg; and 0.01 mg to 100 mg/kg.

The conjugated cannabinoids compounds of the present invention are useful for a variety of therapeutic applications. For example, the compounds are useful for treating or alleviating symptoms of diseases and disorders involving CB1, CB2, GPR119, 5HT_{1A}, μ and δ-OPR receptors, and TRP channels, including appetite loss, nausea and vomiting, pain, multiple sclerosis, and epilepsy. For example, they may be used to treat pain (i.e. as analgesics) in a variety of applications including but not limited to pain management. In additional embodiments, such conjugated cannabinoids may be used as an appetite suppressant. Additional embodiments may include administering the conjugated cannabinoids compounds.

By “treating,” the present inventors mean that the compound is administered in order to alleviate symptoms of the disease or disorder being treated. Those of skill in the art will recognize that the symptoms of the disease or disorder that is treated may be completely eliminated or may simply be lessened. Further, the compounds may be administered in combination with other drugs or treatment modalities, such as with chemotherapy or other cancer-fighting drugs.

Implementation may generally involve identifying patients suffering from the indicated disorders and administering the compounds of the present invention in an acceptable form by an appropriate route. The exact dosage to be administered may vary depending on the age, gender, weight, and overall health status of the individual patient, as well as the precise etiology of the disease. However, in general, for administration in mammals (e.g. humans), dosages in the range of from about 0.01 to about 300 mg of compound per kg of body weight per 24 hr, and more preferably about 0.01 to about 100 mg of compound per kg of body weight per 24 hr, may be effective. Administration may be oral or parenteral, including intravenously, intramuscularly, subcutaneously, intradermal injection, intraperitoneal injection, etc, or by other routes (e.g. transdermal, sublingual, oral, rectal, and buccal delivery, inhalation of an aerosol, etc.). In a preferred embodiment of the invention, the conjugated cannabinoids are provided orally or intravenously.

The compounds may be administered in the pure form or in a pharmaceutically acceptable Formulation including suitable elixirs, binders, and the like (generally referred to as a “secondary carrier”) or as pharmaceutically acceptable salts (e.g. alkali metal salts such as sodium, potassium, calcium or lithium salts, ammonium, etc.) or other complexes. It should be understood that the pharmaceutically acceptable Formulations include liquid and solid materials conventionally utilized to prepare both injectable dosage forms and solid dosage forms such as tablets and capsules and aerosolized dosage forms. In addition, the compounds may be Formulated with aqueous or oil-based vehicles. Water may be used as the carrier for the preparation of compositions (e.g. injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc). Solid diluents and excipients include lactose, starch, conventional disintergrating agents, coatings, and the like. Preservatives such as methyl paraben or benzalkium chloride may also be used. Depending on the Formulation, it is expected that the active composition will consist of about 1% to about 99% of the composition and the secondary carrier will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, bolus dose, or at a gradual or continuous, constant, or controlled rate to a patient. In addition, the time of day and the number of times per day that the pharmaceutical Formulation is administered may vary and are best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities.

As used herein the singular forms “a,” “an,” and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth. Similarly, the word “of” is intended to include “and” unless the context clearly indicates otherwise. Hence “comprising A or B” means including A, or B, or A and B. Furthermore, the use of the term “including,” as well as other related forms, such as “includes” and “included,” is not limiting. The term “about” as used herein is a flexible word with a meaning similar to “approximately” or “nearly.” The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

### Cannabinoid Pro-Drug Synthesis Scheme 1

In one embodiment, the present invention provides for the synthesis of Formula I, where R⁴ is amino acids and R⁵ is inorganic or organic bases: As described below, in one embodiment, CBD can be conjugated with glycine and its salts:

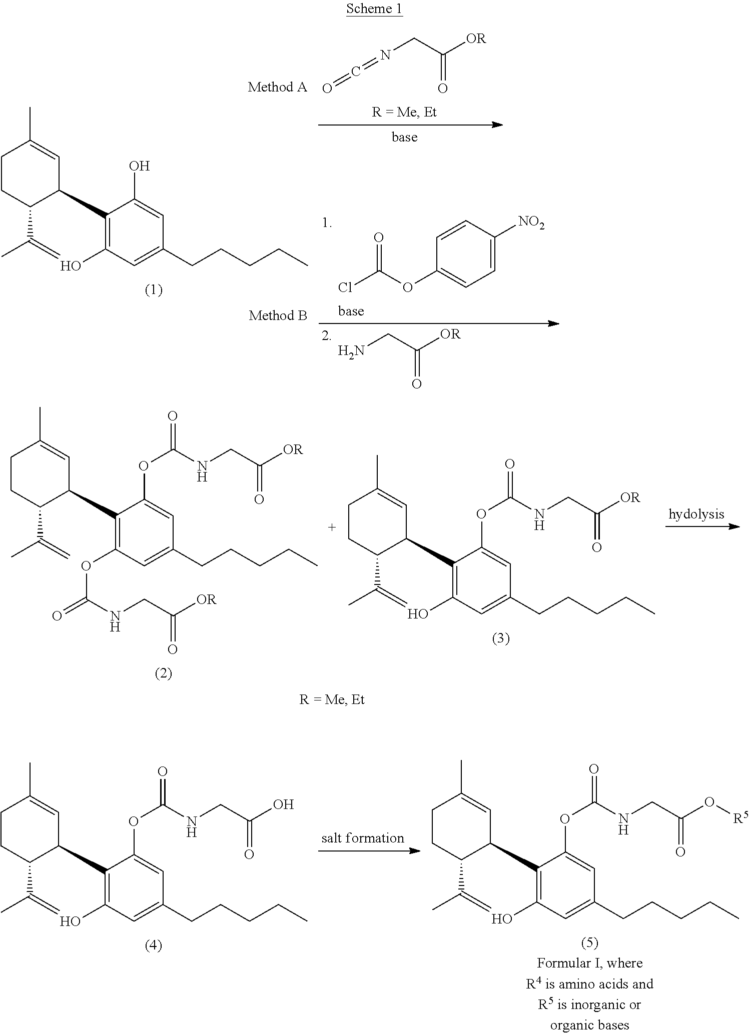

Compounds (5) of Formula I, where R⁴ represents amino acids and R⁵ represents inorganic or organic bases, are prepared using two methods. Method A involves coupling CBD (1) with readily available 2-isocyanatoacetate to produce CBD bis-glycine carbamate ester (2) as the major product and mono-derivative (3) as the minor product. Both (2) and (3) yield CBD mono-glycine carbamate (4) under basic hydrolysis conditions, which then are converted into various salt forms (5) (Scheme 1). Method B employs p-nitrophenyl chloroformate activation. Carbonate intermediates generated by the reaction of CBD (1) with p-nitrophenyl chloroformate react with glycine ester as a nucleophile to produce compounds (2) and (3). Alternatively, glycine methyl ester may be activated by p-nitrophenyl chloroformate and CBD (1) can be used as a nucleophile to yield identical products (2) and (3). Other activating agents such as bis(pentafluorophenyl) carbonate, N,N-disuccinimidyl carbonate, carbonyl diimidazole (CDI), diphosgene (DP), or triphosgene (TP) may also be used for the coupling reactions.

For salt formation, inorganic and organic bases are used: lithium hydroxide, lithium carbonate, sodium hydroxide, sodium bicarbonate, potassium hydroxide, potassium carbonate, calcium hydroxide, calcium carbonate, magnesium hydroxide, magnesium carbonate, lysine, arginine, histidine, diethylaminoethanol, Tris base, meglumine, and the like.

### Cannabinoid Pro-Drug Synthesis Scheme 2

In one embodiment, the present invention provides for the synthesis of Formula III, where R⁴ is amino acids and R⁵ is inorganic or organic bases. As described below, in one embodiment, CBG can be conjugated with glycine and its salts:

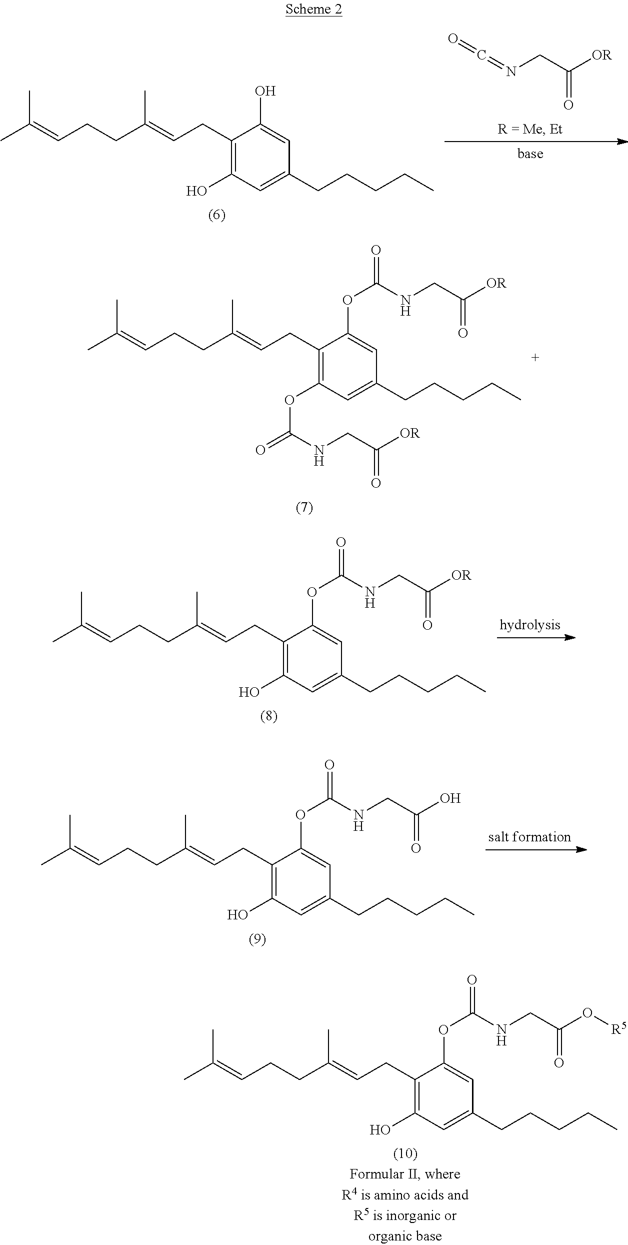

Compounds (10) of Formula III are prepared using a similar method to that described in Scheme 1.

### Cannabinoid Pro-Drug Synthesis Scheme 3

In one embodiment, the present invention provides for the synthesis of Formula IV, where R³ is amino acids and R⁴ is inorganic or organic bases. As described below, in one embodiment, CBN can be conjugated with GABA and its salts:

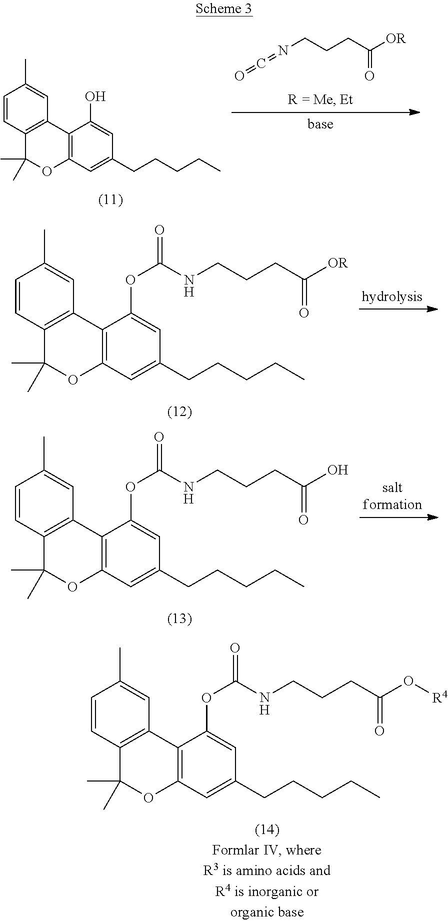

Compounds (14) of Formula IV are prepared following a similar method to that described in Scheme 1 using known 4-isocyanatobutanoate (WO2015077502; Kim, I. -H. et al. J. Med. Chem. 2005, 48, 3621, both of which are incorporated herein by reference) instead of 2-isocyanatoacetate.

### Cannabinoid Pro-Drug Synthesis Scheme 4

In one embodiment, the present invention provides for the synthesis of Formula IV, where R³ is aminosulfonic acid derivatives and R⁴ is inorganic or organic bases. As described below, in one embodiment, CBN can be conjugated with taurine and its salts:

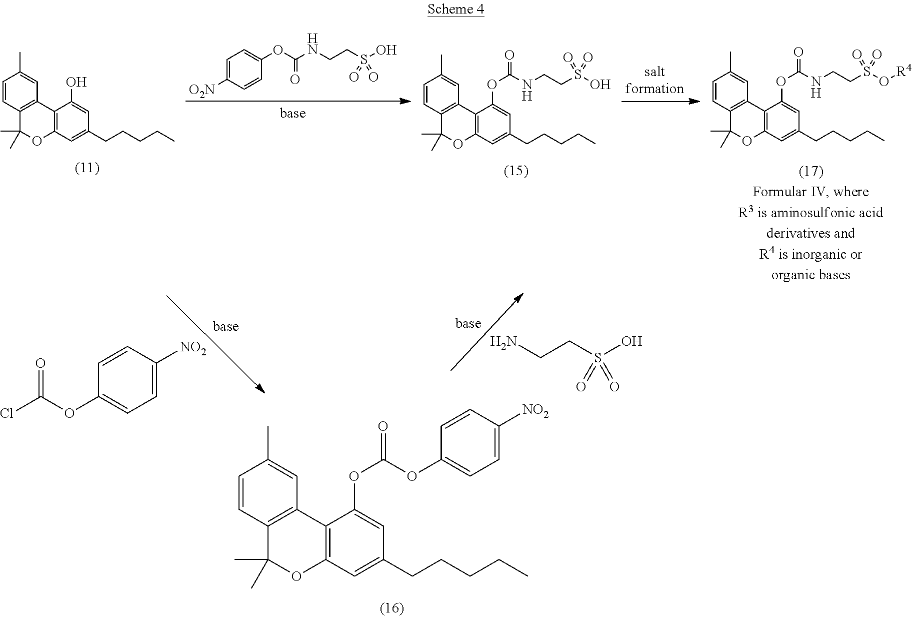

Compound (15) is prepared by reacting CBN (11) with either known 2-(((4-nitrophenoxy)carbonyl)amino)ethane-1-sulfonic acid (Besret, Soizic et al. Bioconjugate Chemistry 2014, 25, 1000, incorporated herein by reference), or by reacting carbonate intermediates (16) generated by the reaction of CBN (11) with taurine. Finally, compound (17) of Formula IV is produced by forming a salt of compound (15), where R3 represents aminosulfonic acid derivatives and R4 represents inorganic or organic bases.

### Cannabinoid Pro-Drug Scheme 5

In one embodiment, the present invention provides for the synthesis of Formula IV, where R³ is amino sugars. As described below, in one embodiment, CBN can be conjugated with meglumine:

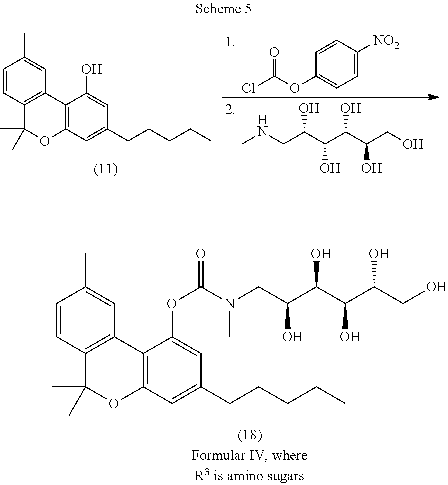

Compounds (18) of formula IV are prepared following a similar method to that described in Scheme 4 using meglumine as the nucleophile.

The preparation of mono-substituted CBD conjugate (4) and CBG conjugate (9) is shown in Scheme 6. Under basic hydrolysis conditions, one of the carbamate ester groups in compounds (2) and (7) is selectively hydrolyzed to produce valuable mono-substituted conjugates (4) and (9) (Examples 2 and 8). These mono-substituted conjugates have value not only in pharmaceutical and consumer product applications but also in their versatile synthetic utility. For example, conjugate (4) can be used as a starting material for the efficient synthesis of THC conjugate (19) and THC (20).

### Cannabinoid Pro-Drug Scheme 6

In one embodiment, the present invention provides for the formation of mono-substituted CBD conjugate (4) and CBG conjugate (9) by selective carbamate hydrolysis.

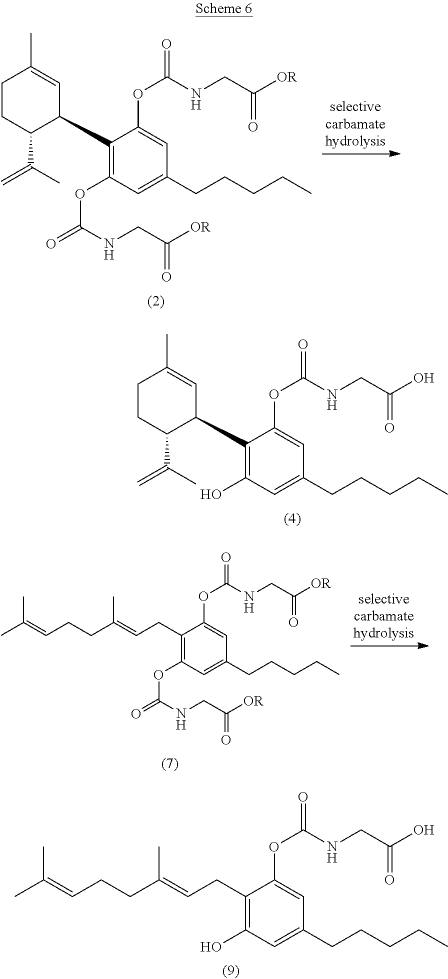

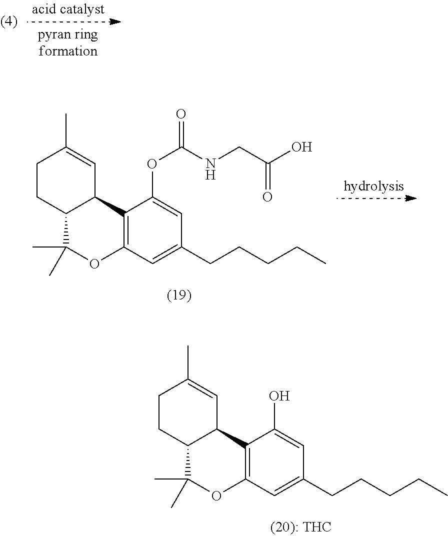

### Scalable and Chromatography-free Synthesis of Compound (5) of Formula I

In one embodiment, a scalable and chromatography-free synthetic route for compound (5) of Formula I is described (Scheme 7). This synthetic route, which modifies the route as outlined in Scheme 1, eliminates the need for chromatography purification of carbamate esters (2) and (3). It also enhances the hydrolysis step. Salting formation with N,N-dicyclohexylmethylamine is employed for the purification of the crude CBD glycine (4), resulting in a good overall yield.

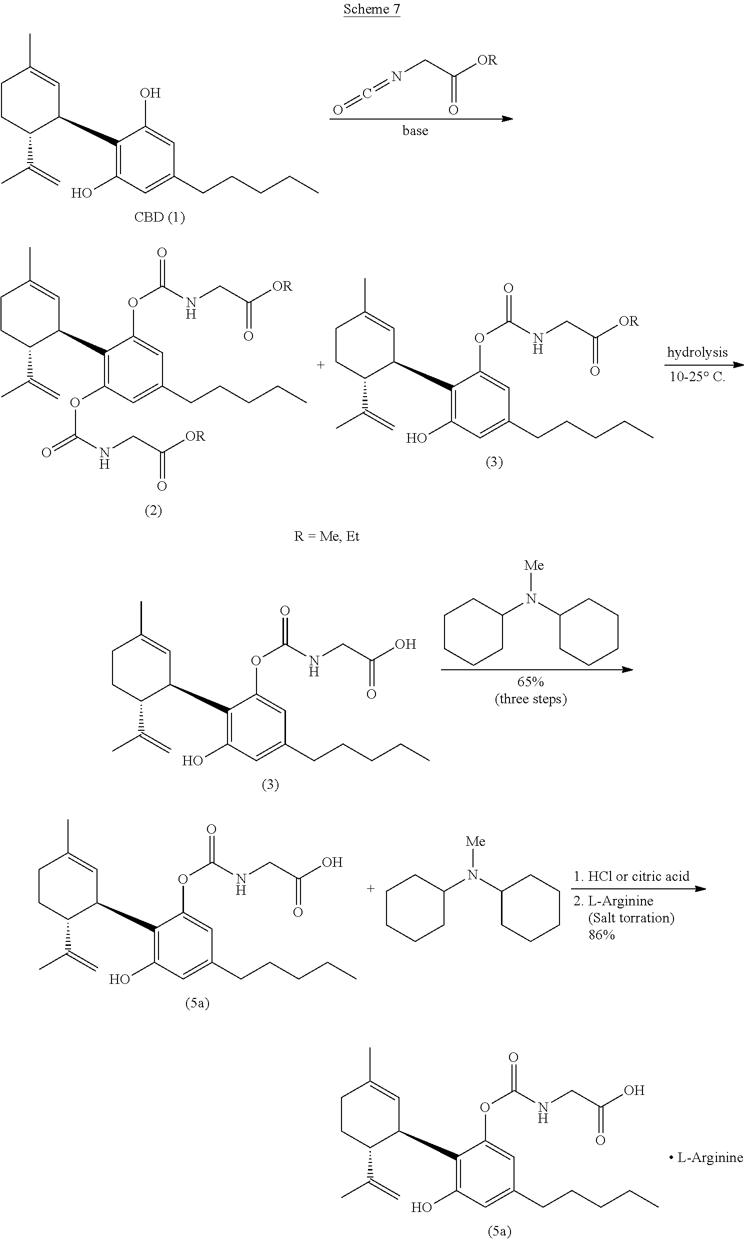

The synthetic route is a four-step process, which begins with the coupling reaction between CBD (1) and ethyl or methyl 2-isocyanatoacetate, similar to the route used in Scheme 1. This results in a mixture of CBD bis-glycine carbamate ester (2) and mono-derivative (3). Instead of purifying them by silica gel column chromatography, the mixture undergoes hydrolysis, yielding exclusively CBD mono-glycine carbamate (4) in the form of sticky oil. Crude (4) is then purified through salt formation with N,N-dicyclohexylmethylamine, leading to compound (5a) as a white solid. This process achieves over 60% yield (three steps) with >97% HPLC purity. Other commercially available amines such as dicyclohexylamine, 1-phenylethylamine, 1,2,3,4-tetrahydro-1-naphthylamine, 1-phenylpiperazine, N-benzyl-t-butylamine, or quinine can also be used in the salt formation. Lastly, N,N-dicyclohexylmethylamine salt (5a) can be converted into various pharmaceutical or consumable grade salt forms. This involves acidification and salt formation. The formation of arginine CBD glycinate salt (5b) is shown as a representative example, which can be achieved in methanol without using water (Examples 15-18).

### Cannabinoid Prodrug Synthesis of Formula II

In one embodiment, the present invention provides for the synthesis of a compound according to Formula II, where R³ is amino acids and R⁴ is inorganic or organic bases. As described below, in one embodiment, THCV can be conjugated with glycine and its salts:

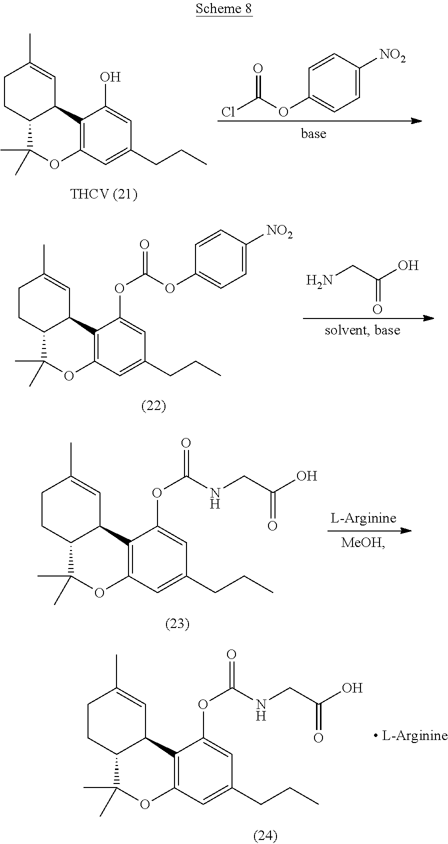 [SMILES: CCCc1cc(O)c2c(c1)OC(C)(C)[C@@H]1CCC(C)=C[C@@H]21 ; O=C(Cl)Oc1ccc([N+](=O)[O-])cc1 ; CCCc1cc(OC(=O)Oc2ccc([N+](=O)[O-])cc2)c2c(c1)OC(C)(C)[C@@H]1CCC(C)=C[C@@H]21 ; NCC(=O)O ; CCCc1cc(OC(=O)NCC(=O)O)c2c(c1)OC(C)(C)[C@@H]1CCC(C)=C[C@@H]21 ; CCCc1cc(OC(=O)NCC(=O)O)c2c(c1)OC(C)(C)[C@@H]1CCC(C)=C[C@@H]21]

The synthesis of compound (24) of Formula II can be achieved similarly to Scheme 4 (Scheme 8). Carbonate intermediate (22), prepared by reacting THCV (21) with 4-nitrophenyl chloroformate, is treated with glycine in t-BuOH or t-amyl alcohol in the presence of a base. This affords the THCV glycine (23), which converts into arginine THCV glycinate salt (24) (Examples 19-21). Instead of using 4-nitrophenyl chloroformate, other activating agents, such as bis(pentafluorophenyl) carbonate, N,N-disuccinimidyl carbonate, carbonyl diimidazole (CDI), diphosgene (DP), or triphosgene (TP), may also be used for the coupling reactions.

### Scalable and Chromatography-free Synthesis of Compound (10) of Formula III

In one embodiment, a scalable process for compound (10) of Formula III is described (Scheme 9), similar to Scheme 7, which is based on the synthetic route shown in Scheme 2. The synthesis begins with the coupling reaction of CBG (6) with ethyl or methyl 2-isocyanatoacetate, resulting in a mixture of bis- and mono-carbamates (7) and (8). Hydrolysis of crude products (7) and (8) give exclusively CBG mono-glycine carbamate (9). The purification of (9) can be achieved by the salt formation with 1,2,3,4-tetrahydro-1-naphthylamine, producing 1,2,3,4-tetrahydro-1-naphthylamine CBG glycinate salt (10a) as a white solid. Other amines such as dicyclohexylamine, N,N-dicyclohexylmethylamine, 1-phenylethylamine, 1-phenylpiperazine, N-benzyl-t-butylamine, or quinine can also be used. Compound (10a) is then transformed into various pharmaceutical or consumable grade salt forms, involving acidification and salt formation. Formation of arginine CBG glycinate (10b) is shown as a representative example, which is achieved in methanol without using water (Examples 22-25).

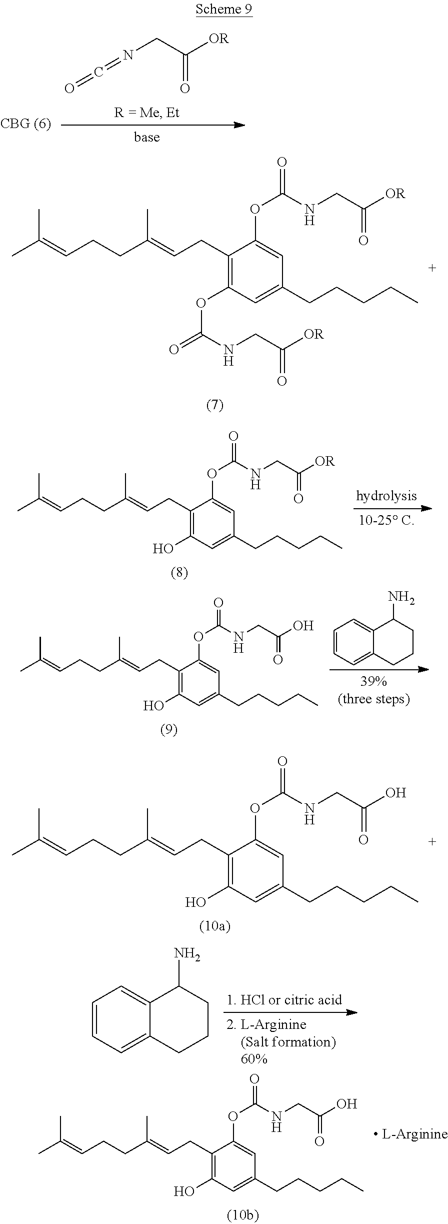

### Scalable and Chromatography-free Synthesis of Compound (14) of Formula IV

In one embodiment, a new synthetic route for a large-scale production of compound (14) of Formula IV is provided, modifying the synthetic route described in Scheme 3 (Scheme 10). The synthesis begins with a direct coupling of carbonate intermediate (16) with y-aminobutyric acid (GABA) in the presence of base such as NEt3. Several solvents including t-amyl alcohol, t-butanol, DMF, trifluoroethanol, or hexafluoroisopropanol are effective for this conversion Among them, t-amyl alcohol and t-butanol works best. Crude CBN y-aminobutyric acid (GABA) (13) is then purified by salt formation with 1-phenylethylamine and isolated as a white solid. Other amines such as dicyclohexylamine, N,N-dicyclohexylmethylamine, 1,2,3,4-tetrahydro-1-naphthylamine, 1-phenylpiperazine, N-benzyl-t-butylamine, or quinine can be also used for the purification process. Lastly, 1-phenylethylamine CBD y-aminobutyrate salt (14a) is converted arginine CBN y-aminobutyrate salt (14b) (Examples 26-29).

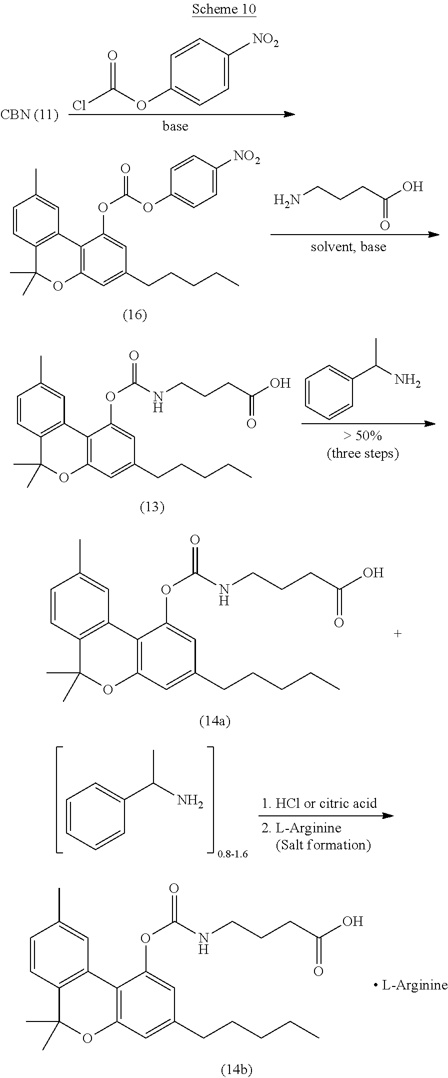

### Cannabinoid Prodrug Synthesis of Formula (I) Using Isocyanate Equivalents or in Situ Generated Isocyanates

In one embodiment, the present invention provides for the synthesis of Formula 1, where R⁴ is amino acids and R⁵ is inorganic or organic bases. As described below, in one embodiment, CBD can be conjugated with glycine and its salts:

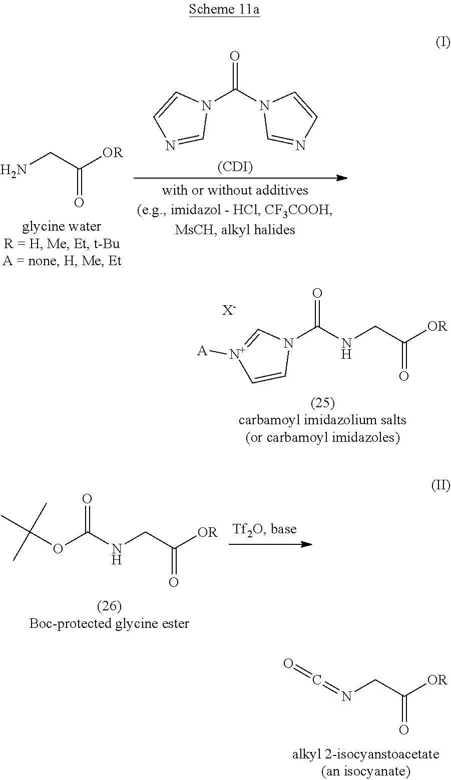

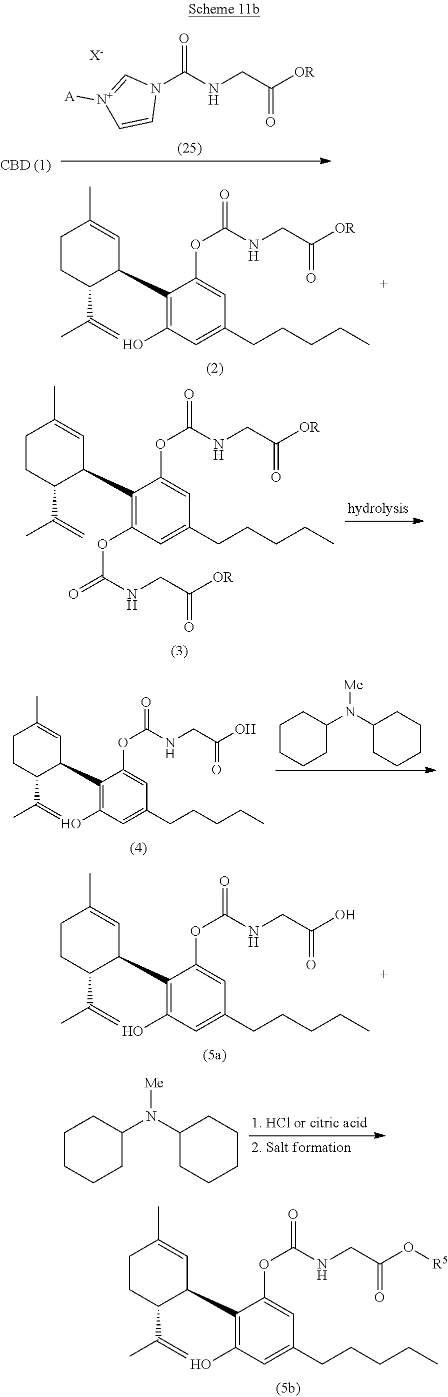

In Scheme 1, compound (5) from Formula I is prepared by coupling CBD (1) with ethyl (or methyl) 2-isocyanatoacetate. Instead of using the isocyanate directly, isocyanate equivalents or in situ generated isocyanates can also be effectively used for the same coupling reaction (Scheme 11a). During the process of generating isocyanate equivalents, the glycine ester is activated to produce reactive carbamoyl imidazolium salts (or carbamoyl imidazole) (25) in situ. This then reacts with CBD to yield a mixture of CBD bis-glycine carbamate ester (2) and mono-derivative (3) (Scheme 11b). The use of additives, including Bronsted acids such as HCl, trifluoroacetic acid, or methanesulfonic acid (MsOH), as well as alkylating agents is recognized for its ability to increase the reaction rate and reduce the formation of side products (J. Org. Chem. 2022, 87, 11329-11349; Org. Process Res. Dev. 2021, 25,500-506; Org. Process Res. Dev. 2009, 13,106-113). The remaining steps can be carried out in the same manner as described in Scheme 7 (Examples 32-34).

To generate in situ generated isocyanates, for instance, ethyl (or methyl) 2-isocyanatoacetate, a Boc-protected glycine ester can be treated with triflic anhydride (Tf2O) in the presence of base such as 2-chloropyridine according to the literature (J. Org. Chem. 20214, 79, 4477-4483). This in situ generated isocyanate can be used for the coupling reaction with CBD.

### Cannabinoid Prodrug Synthesis of Formula (I) Using Carbonyl diimidazole (CDI) as an Activating Agent

In one embodiment, the present invention provides for the synthesis of Formula 1, where R4 is amino acids and R5 is inorganic or organic bases. As described below, in one embodiment, CBD can be conjugated with γ-aminobutyric acid (GABA) and its salts:

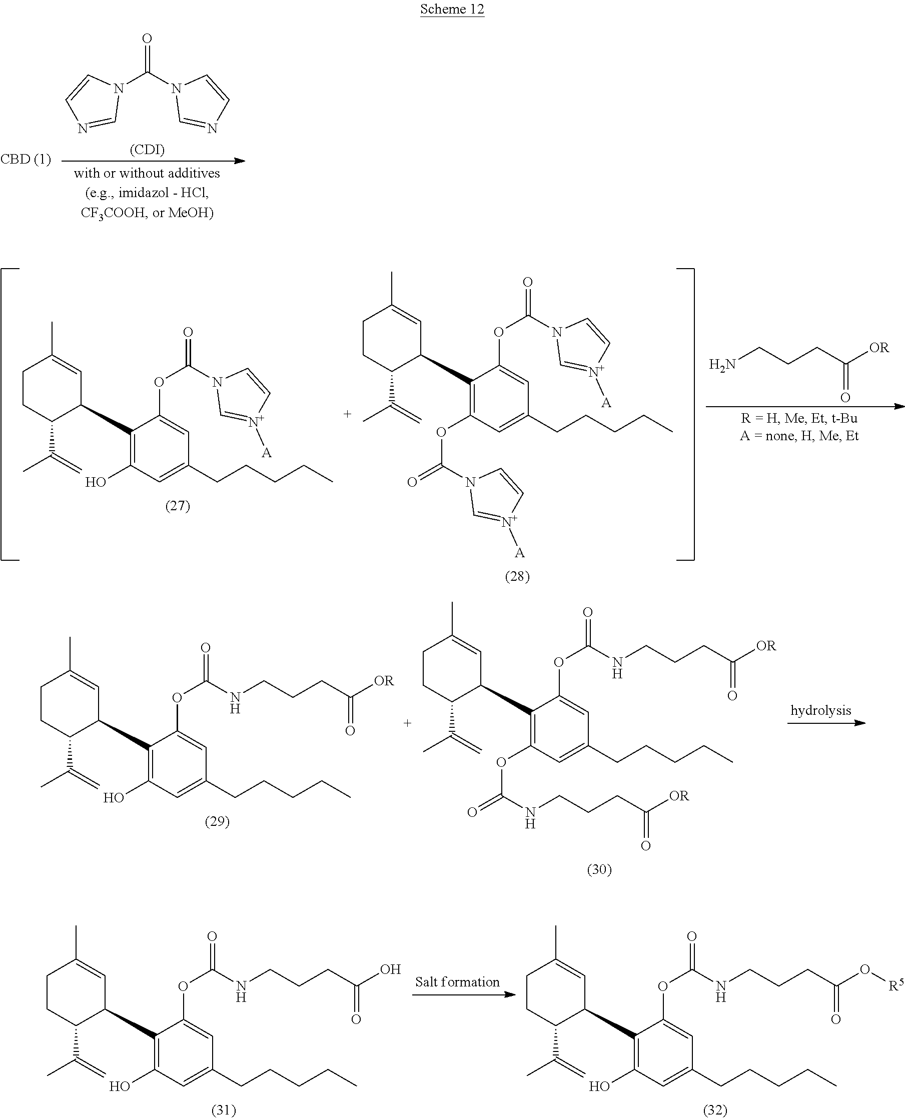

Schemes I (Method B), 4, and 5 have demonstrated that the cannabinoid system can be activated by treatment of 4-nitrophenyl chloroformate before coupling with amine partners. Similarly, CBD (1) can also be activated using carbonyl diimidazole (CDI), where additives such as Bronsted acids like HCl, trifluoracetic acid, or methanesulfonic acid (MsOH) can further enhance the reactivity profiles and reduce the formation of side products (Scheme 12). Consequently, the resulting acyl imidazolium (or imidazole intermediates) (27-28) reacts with γ-aminobutyric ester (GABA ester) to produce a mixture of CBD bis-γ-aminobutyric acid carbamate ester (29) and mono-derivative (30). The remaining steps can be carried out in the similar manner as described in Scheme 7 (Examples 35-37).

The compounds described in this invention may be synthesized using various methods, including standard chemistry. Schemes 1-6 that serve as examples are provided below, followed by the preparation of specific compounds in the Examples section. Any variable that has been defined previously will retain its designated meaning unless stated otherwise.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### General Procedures

All solvents and reagents were purchased from commercial sources and used without further purification unless otherwise noted. Reactions were monitored by Thin Layer Chromatography (TLC) on 0.25 mm silica gel 60 F254 plates from MilliporeSigma, visualized using UV light, basic aqueous potassium permanganate (KMnO4), or iodine staining. EMD Millipore silica gel (60 Å, particle size 63-200 μm) was used for column chromatography. All NMR spectra were recorded at 300 K on Bruker Ultrashield 300 or 500 MHz spectrometer, calibrated using residual undeuterated solvent as an internal reference. NMR data were processed using Mnova NMR processing software (Mestrelab Research S.L.). Analytical HPLC were performed on a Shimadzu 2050C using a Raptor ARC-18 column (100 mm×4.6 mm×5 μm) with a gradient with (1) acetonitrile with 0.1% formic acid and (2) water with 0.1% formic acid and 5 mM ammonium formate at a flow rate of 1.5 mL/min. Detection was monitored at 220 nm.

### Example 1: Synthesis of Diethyl 2,2′-(((((1′R,2′R)-5′-methyl-4-pentyl-2′-(prop-1-en-2-yl)-1′,2′,3′,4′-tetrahydro-[1,1′-biphenyl]-2,6-diyl)bis(oxy))bis(carbonyl))bis(azanediyl))diacetate and ethyl ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate

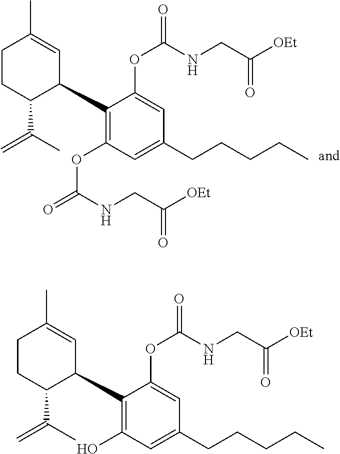 [SMILES: C=C(C)[C@@H]1CCC(C)=C[C@H]1c1c(OC(=O)NCC(=O)OCC)cc(CCCCC)cc1OC(=O)NCC(=O)OCC ; C=C(C)[C@@H]1CCC(C)=C[C@H]1c1c(O)cc(CCCCC)cc1OC(=O)NCC(=O)OCC]

### Method A

To a solution of CBD (5.0 g, 0.02 mol) in 2-MeTHF (20 mL) was treated with ethyl 2-isocyanatoacetate (5.0 g, 0.03 mol, 2.2 equiv), followed by triethylamine (6.0 mL, 0.05 mol, 2.9 equiv). The mixture was stirred at 55° C. for 10 h. Upon completion, the mixture was diluted with iPrOAc (60 mL), washed with water, brine, dried (Na2SO4), filtered, and concentrated under reduced pressure. The crude was purified by silica gel column chromatography (SiO2, 208 g; Eluent, 10% EtOAc in hexane to 50% EtOAc) to give diethyl 2,2′-(((((1′R,2′R)-5′-methyl-4-pentyl-2′-(prop-1-en-2-yl)-1′,2′,3′,4′-tetrahydro-[1,1′-biphenyl]-2,6-diyl)bis(oxy))bis(carbonyl))bis(azanediyl))diacetate (7.68 g, 80%) as a major product. ¹H NMR (300 MHz, CD3OD): δ 6.75 (s, 2H), 5.16 (s, 1H), 4.55-4.42 (m, 2H), 4.21 (q, J=7.1 Hz, 4H), 3.91 (dd, J=18.2, 17.8 Hz, 4H), 3.65 (d, J=10.8 Hz, 1H), 2.82 (td, J=10.6, 4.5 Hz, 1H), 2.62-2.50 (m, 2H), 2.40-2.22 (m, 1H), 1.99 (d, J=19.5 Hz, 1H), 1.81-1.71 (m, 2H), 1.71-1.53 (m, 8H), 1.44-1.20 (m, 10H), 0.97-0.83 (m, 3H). And ethyl ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate (961 mg, 10%) was obtained as a minor product.

### Method B

To a solution of CBD (1000 mg, 3.180 mmol) in DCM (8 mL) was treated with 4-nitrophenyl chloroformate (1.282 g, 6.360 mmol, 2.0 equiv) in DCM (5 mL) at 0° C., followed by addition of triethylamine (886 μL, 6.36 mmol, 2.0 equiv). After the mixture was stirred at rt for 30 min, a solution of glycine methyl ester hydrochloride (798.5 mg, 6.360 mmol, 2.0 equiv) and triethylamine (443 μL, 3.18 mmol, 1.0 equiv) in DCM (8 mL) was added. The mixture was stirred at rt for overnight. The mixture was diluted with DCM, washed with satd NaHCO3 solution, brine, dried (Na2SO4), filtered, and concentrated. The crude product was purified by column chromatography (SiO2, 40 g; Eluent, 5% iPrOAc to 30% iPrOAc in hexane to give diethyl 2,2′-(((((1′R,2′R)-5′-methyl-4-pentyl-2′-(prop-1-en-2-yl)-1′,2′,3′,4′-tetrahydro-[1,1′-biphenyl]-2,6-diyl)bis(oxy))bis(carbonyl))bis(azanediyl))diacetate (616 mg, 35%) and ethyl ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate (819 mg, 60%).

### Example 2: Synthesis of ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycine

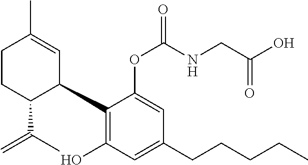 [SMILES: C=C(C)[C@@H]1CCC(C)=C[C@H]1c1c(O)cc(CCCCC)cc1OC(=O)NCC(=O)O]

Diethyl 2,2′-(((((1′R,2′R)-5′-methyl-4-pentyl-2′-(prop-1-en-2-yl)-1′,2′,3′,4′-tetrahydro-[1,1′-biphenyl]-2,6-diyl)bis(oxy))bis(carbonyl))bis(azanediyl))diacetate (3.971 g, 6.934 mmol) was dissolved in methanol (31 mL) and a solution of lithium hydroxide monohydrate (611.0 mg, 14.56 mmol, 2.1 equiv) in water (25 mL) at 0-10° C. was added to the solution. The mixture was stirred for 10-20 min at rt. Methanol was removed under reduced pressure (bath 35° C.), and water (20 mL) was added to the residue. The aqueous layer was extracted with MTBE (30 mL). The organic layer was dried (Na2SO4), filtered, and concentrated to give target product in an impure form (2.194 g, 76%). A second crop of the pure product was obtained by adjusting the pH of the aqueous layer to 3-4 with citric acid (2.331 g, 12.13 mmol, 1.75 equiv). The product was then extracted with MTBE (40 mL X 1 and 20 mL X 1). The combined organic layers were washed with brine (5 mL), dried (Na2SO4), filtered, and concentrated to afford the title compound (680 mg, 23%), which was used for the next salt formation. 1H NMR (300 MHz, CD3OD): δ 6.44 (s, 1H), 6.33 (s, 1H), 5.19 (s, 1H), 4.48 (d, J=1.5 Hz, 1H), 4.44 (d, J=1.5 Hz, 1H), 4.06-3.71 (m, 3H), 2.88-2.82 (m, 1H), 2.46 (t, J=7.7 Hz, 2H), 2.28-2.22 (m, 1H), 1.96 (d, J=19.0 Hz, 1H), 1.79-1.68 (m, 2H), 1.68-1.50 (m, 8H), 1.37-1.26 (m, 4H), 0.90 (t, J=6.8 Hz, 3H).

### Example 3: Synthesis of Sodium ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate

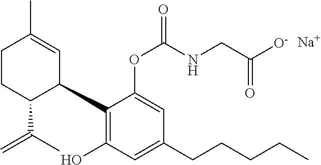 [SMILES: C=C(C)[C@@H]1CCC(C)=C[C@H]1c1c(O)cc(CCCCC)cc1OC(=O)NCC(=O)[O-] ; [Na+]]

((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycine (670 mg, 1.30 mmol) was dissolved in methanol (7.9 mL) and a solution of sodium bicarbonate (1.09 g, 13.0 mmol, 10 equiv) in water (9.35 mL) was added 5 to the solution at rt. The mixture was stirred at rt for 3 h. After methanol had been removed, water (15 mL) was added to the residue. The product was extracted with MTBE (40 mL), washed with brine (4 mL), dried (Na2SO4), filtered and concentrated in vacuo. The residue was triturated with heptane to afford the title compound as an off-white solid (414 mg, 59%).1H NMR (300 MHz, CD3OD): δ 6.43 (s, 1H), 6.37 (s, 1H), 5.20 (s, 1H), 4.45 (s, 1H), 4.43 (s, 1H), 3.87 (m, 1H), 3.78 (d, J=17.0 Hz, 1H), 3.62 (d, J=17.2 Hz, 1H), 2.83 (m, 1H), 2.46 (t, J=7.7 Hz, 2H), 2.26 (s, 1H), 1.95 (d, J=17.4 Hz, 1H), 1.80-1.69 (m, 2H), 1.69-1.50 (m, 8H), 1.49-1.21 (m, 4H), 0.90 (t, J=6.7 Hz, 3H).

### Example 4: Synthesis of L-Lysine ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate

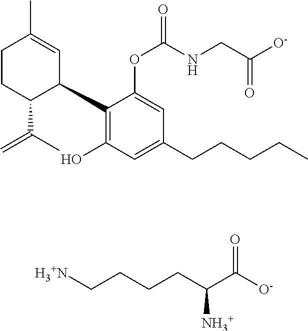

The title compound (123 mg, 61%) was prepared following a similar method as described in Example 3 using ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycine (150 mg, 0.361 mmol) and L-lysine (90 mg, 0.614 mmol) in methanol and water. 1H NMR (300 MHz, CD3OD): δ 6.44 (s, 1H), 6.39 (s, 1H), 5.19 (s, 1H), 4.48-4.40 (m, 2H), 3.90-3.84 (m, 1H), 3.78 (d, J=16.8 Hz, 1H), 3.61 (d, J=17.2 Hz, 1H), 3.47 (t, J=6.0 Hz, 1H), 2.88 (m, 3H), 2.46 (t, J=7.7 Hz, 2H), 2.28-2.22 (m, 1H), 1.99-1.91 (m, 1H), 1.88-1.55 (m, 14H), 1.52-1.46 (m, 2H), 1.36-1.30 (m, 4H), 0.90 (t, J=6.7 Hz, 3H).

### Example 5: Synthesis of L-Arginine ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate

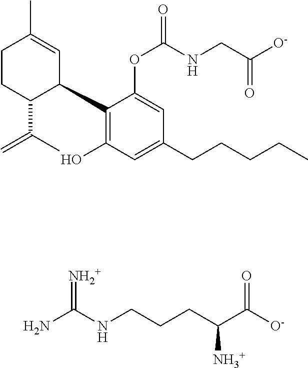 [SMILES: C=C(C)[C@@H]1CCC(C)=C[C@H]1c1c(O)cc(CCCCC)cc1OC(=O)NCC(=O)[O-] ; NC(=[NH2+])NCCC[C@H]([NH3+])C(=O)[O-]]

The title compound (174 mg, 61%) was prepared following a similar method as described in Example 3 using ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycine (210 mg, 0.505 mmol) and L-arginine (70 mg, 0.455 mmol) in methanol and water. 1H NMR (300 MHz, CD3OD): δ 6.44 (s, 1H), 6.39 (s, 1H), 5.18 (s, 1H), 4.49-4.37 (m, 2H), 3.99-3.83 (m, 1H), 3.78 (d, J=17.2 Hz, 1H), 3.68-3.50 (m, 2H), 3.20 (t, J=6.9 Hz, 2H), 2.94-2.73 (m, 1H), 2.46 (t, J=7.7 Hz, 2H), 2.37-2.17 (m, 1H), 2.06-1.47 (m, 17H), 1.43-1.18 (m, 4H), 0.90 (t, J=6.7 Hz, 3H).

### Example 6: Synthesis of Meglumine ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycinate

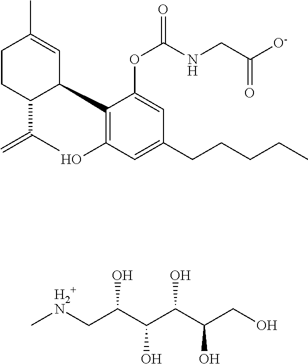

The title compound (217 mg, 59%) was prepared following a similar method as described in Example 3 using ((((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)carbonyl)glycine (250 mg, 0.602 mmol) and meglumine (106 mg, 0.541 mmol) in methanol and water. 1H NMR (300 MHz, CD3OD): δ 6.43 (d, J=1.7 Hz, 1H), 6.39 (s, 1H), 5.19 (s, 1H), 4.53-4.34 (m, 2H), 4.11-3.98 (m, 1H), 3.94-3.55 (m, 8H), 3.24-3.08 (m, 2H), 2.91-2.77 (m, 1H), 2.69 (s, 3H), 2.46 (t, J=7.7 Hz, 2H), 2.36-2.16 (m, 1H), 1.95 (d, J=17.1 Hz, 1H), 1.82-1.49 (m, 10H), 1.42-1.25 (m, 4H), 0.90 (t, J=6.7 Hz, 3H).

### Example 7: Synthesis of Diethyl 2,2′-((((2-(3,7-dimethylocta-2,6-dien-1-yl)-5-pentyl-13-phenylene)bis(oxy))bis(carbonyl))bis(azanediyl))(E)-diacetate and ethyl (E)-((2-(3,7-dimethylocta-2,6-dien-1-yl)-3-hydroxy-5-pentylphenoxy)carbonyl)glycinate

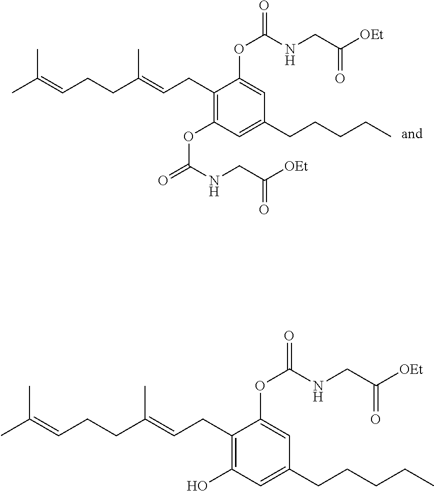

To a solution of CBG (1163 mg, 3.675 mmol) in 2-MeTHF (26 mL) was treated with ethyl 2-isocyanatoacetate (996 mg, 7.717 mmol, 2.1 equiv), followed by triethylamine (1.13 mL, 8.09 mmol, 2.2 equiv). The mixture was stirred at 55° C. for 10 h. Upon completion, the mixture was diluted with iPrOAc (60 mL), washed with water, brine, dried (Na2SO4), filtered, and concentrated under reduced pressure. The crude was purified by silica gel column chromatography (SiO2, 30 g; Eluent, 3% iPrOAc in heptane to 30% iPrOAc in heptane) to give Diethyl 2,2′-((((2-(3,7-dimethylocta-2,6-dien-1-yl)-5-pentyl-1,3 phenylene)bis(oxy))bis(carbonyl))bis(azanediyl))(E)-diacetate_(1.96 g, 92%). 1H NMR (300 MHz, CD3OD): δ 6.82 (s, 2H), 5.23-4.99 (m, 2H), 4.21 (q, J=7.0 Hz, 4H), 3.91 (s, 4H), 3.26 (d, J=7.2 Hz, 2H), 2.58 (t, J=7.7 Hz, 2H), 2.21-1.98 (m, 2H), 1.98-1.86 (m, 2H), 1.74 (d, J=7.4 Hz, 3H), 1.69-1.53 (m, 8H), 1.43-1.32 (m, 4H), 1.28 (t, J=7.0 Hz, 6H), 0.91 (t, J=6.7 Hz, 3H). And ethyl (E)-((2-(3,7-dimethylocta-2,6-dien-1-yl)-3-hydroxy-5-pentylphenoxy)carbonyl)glycinate (0.101 g, 6.2%) was obtained as a minor product.

### Example 8: Synthesis of (E)-((2-(3,7-Dimethylocta-2,6-dien-1-yl)-3-hydroxy-5-pentylphenoxy)carbonyl)glycine

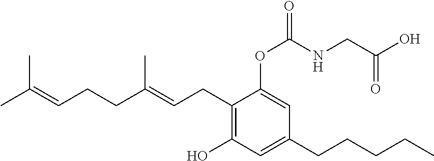 [SMILES: CCCCCc1cc(O)c(C/C=C(\C)CCC=C(C)C)c(OC(=O)NCC(=O)O)c1]

To a solution of diethyl 2,2′-((((2-(3,7-dimethylocta-2,6-dien-1-yl)-5-pentyl-1,3-phenylene)bis(oxy))bis(carbonyl))bis(azanediyl))(E)-diacetate (1638 mg, 2.850 mmol) in methanol (22 mL) was treated with a solution of lithium hydroxide monohydrate (239.0 mg, 5.700 mmol, 2.0 equiv) in water (10 mL) at 0-10° C. The mixture was stirred for 10-20 min at rt. Methanol was removed under reduced pressure (bath 35° C.), and water (30 mL) was added to the residue. The aqueous layer was washed with MTBE (30 mL). The organic layer was dried (Na2SO4), filtered, and concentrated in vacuum give target product in an impure form (1.34 g, 91%). A second crop of the pure product was obtained by adjusting the pH of the aqueous layer to 3-4 with citric acid (821 g, 4.275 mmol, 1.50 equiv). The product was then extracted with MTBE (25 mL×2). The combined organic layers were washed with brine (5 mL), dried (Na2SO4), filtered, and concentrated to afford the target product (334 mg, 28%), which was used for the next salt formation. 1H NMR (300 MHz, CD3OD) δ 6.50 (d, J=1.6 Hz, 1H), 6.39 (d, J=1.6 Hz, 1H), 5.18 (t, J=7.2 Hz, 1H), 5.13-4.99 (m, 1H), 3.88 (s, 2H), 3.24 (d, J=7.1 Hz, 2H), 2.48 (t, J=7.6 Hz, 2H), 2.11-1.99 (m, 2H), 1.99-1.85 (m, 2H), 1.73 (s, 3H), 1.67-1.48 (m, 8H), 1.46-1.23 (m, 4H), 0.90 (t, J=6.7 Hz, 3H).

### Example 9: Synthesis of L-Arginine (E)-((2-(3,7-dimethylocta-2,6-dien-1-yl)-3-hydroxy-5-pentylphenoxy)carbonyl)glycinate

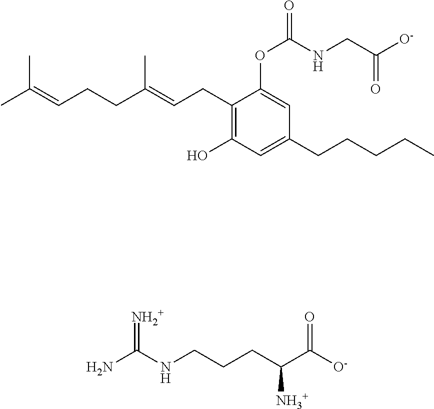 [SMILES: CCCCCc1cc(O)c(C/C=C(\C)CCC=C(C)C)c(OC(=O)NCC(=O)[O-])c1 ; NC(=[NH2+])NCCC[C@H]([NH3+])C(=O)[O-]]

The title compound (163 mg, 69%) was prepared following a similar method as described in Example 3 using (E)-((2-(3,7-dimethylocta-2,6-dien-1-yl)-3-hydroxy-5-pentylphenoxy)carbonyl)glycine (178 mg, 0.343 mmol) and L-arginine (120 mg, 0.686 mmol) in methanol and water. 1H NMR (300 MHz, CD3OD): δ 6.51 (s, 1H), 6.44 (s, 1H), 5.25-5.13 (m, 2H), 3.72 (s, 2H), 3.47 (t, J=5.9 Hz, 1H), 3.28-3.09 (m, 4H), 2.48 (t, J=7.7 Hz, 2H), 2.12-2.00 (m, 2H), 2.00-1.89 (m, 2H), 1.89-1.47 (m, 15H), 1.43-1.24 (m, 4H), 0.91 (q, J=7.3 Hz, 3H).

### Example 10: Synthesis of Sodium (E)-((2-(3,7-dimethylocta-2,6-dien-1-yl)-3-hydroxy-5-pentylphenoxy)carbonyl)glycinate

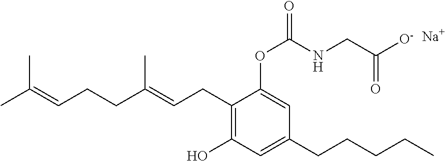 [SMILES: CCCCCc1cc(O)c(C/C=C(\C)CCC=C(C)C)c(OC(=O)NCC(=O)[O-])c1 ; [Na+]]

The title compound (75 mg, 46%) was prepared following a similar manner as described in Example 3 using (E)-((2-(3,7-dimethylocta-2,6-dien-1-yl)-3-hydroxy-5-pentylphenoxy)carbonyl)glycine (156 mg, 0.374 mmol) and sodium bicarbonate (202 mg, 2.40 mmol) in methanol and water. 1H NMR (300 MHz, CD3OD) δ 6.49 (s, 1H), 6.42 (s, 1H), 5.23-5.12 (m, 1H), 5.11-5.00 (m, 1H), 3.75 (s, 2H), 3.24 (d, J=7.1 Hz, 2H), 2.47 (t, J=7.7 Hz, 2H), 2.12-1.99 (m, 2H), 1.99-1.85 (m, 2H), 1.73 (s, 3H), 1.66-1.49 (m, 8H), 1.42-1.20 (m, 4H), 0.91 (t, J=7.2 Hz, 3H).

### Example 11. Ethyl (((6,6,9-trimethyl-3-pentyl-6H-benzo[c]chromen-1-yl)oxy)carbonyl)glycinate

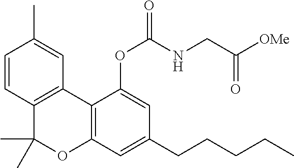 [SMILES: CCCCCc1cc(OC(=O)NCC(=O)OC)c2c(c1)OC(C)(C)c1ccc(C)cc1-2]

The title compound (427 mg, 86%) was prepared following a similar method as described in Example 1 (method A) using CBN (351 mg, 1.13 mmol) and ethyl 2-isocyanatoacetate (161 mg, 1.24 mmol, 1.1 equiv). 1H NMR (300 MHz, CD3OD): δ 7.90 (s, 1H), 7.18 (d, J=7.8 Hz, 1H), 7.10 (d, J=8.1 Hz, 1H), 6.67 (d, J=1.8 Hz, 1H), 6.61 (d, J=1.8 Hz, 1H), 4.17 (q, J=7.1 Hz, 2H), 3.91 (s, 2H), 2.57 (t, J=7.7 Hz, 2H), 2.38 (s, 3H), 1.74-1.58 (m, 2H), 1.59-1.48 (m, 6H), 1.46-1.28 (m, 4H), 1.22 (t, J=7.1 Hz, 3H), 1.00-0.81 (m, 3H).

### Example 12. Methyl 4-((((6,6,9-trimethyl-3-pentyl-6H-benzo[c]chromen-1-yl)oxy)carbonyl)amino)butanoate

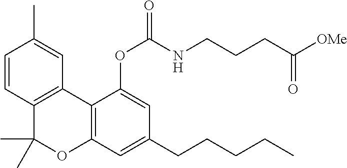 [SMILES: CCCCCc1cc(OC(=O)NCCCC(=O)OC)c2c(c1)OC(C)(C)c1ccc(C)cc1-2]

The title compound (856 mg, 78%) was prepared following a similar method as described in Example 1 (method A) using CBN (750 mg, 2.42 mmol) and readily available methyl 4-isocyanatobutanote (415 mg, 2.90 mmol, 1.2 equiv). TLC (5% iPrOAc in heptane) R_{f}=0.21.

### Example 13. 2-((((6,6,9-trimethyl-3-pentyl-6H-benzo[c]chromen-1-yl)oxy)carbonyl)amino)ethane-1-sulfonic acid

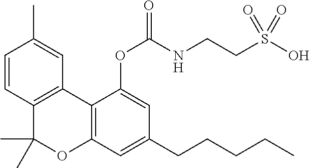 [SMILES: CCCCCc1cc(OC(=O)NCCS(=O)(=O)O)c2c(c1)OC(C)(C)c1ccc(C)cc1-2]

To a solution of CBN (200 mg, 1 Eq, 644 μmol) in dichloromethane (8.3 mL) was treated with p-nitrophenyl chloroformate (143 mg, 709 μmol, 1.1 equiv) at rt, followed by addition of triethylamine (78.2 mg, 108 μL, 1.2 Eq, 773 μmol). The mixture was stirred at rt for 30 min. After concentration, 4-nitrophenyl (6,6,9-trimethyl-3-pentyl-6H-benzo[c]chromen-1-yl) carbonate was dissolved in pyridine (2.08 mL) and a solution of taurine (161 mg, 1.29 mmol, 2.0 equiv) in water (1.04 mL) was added at rt. The mixture was stirred at rt for 2 d and concentrated in vacuum. The crude was purified by column chromatography (DCM only to 10% MeOH in DCM) to give the title compound. TLC (11% MeOH in DCM) R_{f}=0.30.

### Example 14. Solubility of CBD and CBG Glycine Conjugates

The thermodynamic aqueous solubility of CBD and CBG glycine conjugates in salt forms was determined using the equilibrium shake method outlined in the United States Pharmacopeia (USP) General Chapter <1236>Solubility Measurements (Table 1). These compounds exhibited a significantly improved solubility of >22700-fold compared to CBD (0.1 μg/mL) at room temperature.

### Example 15. Synthesis of CBD bis-glycine carbamate ester (2) and mono-derivative (3)

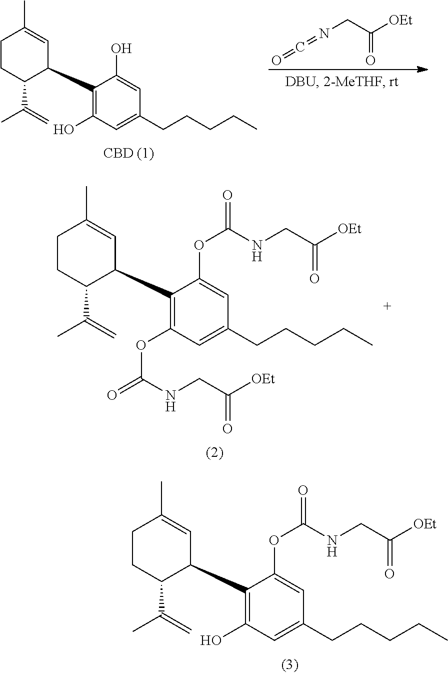 [SMILES: C=C(C)[C@@H]1CCC(C)=C[C@H]1c1c(O)cc(CCCCC)cc1O ; CCOC(=O)CN=C=O ; C=C(C)[C@@H]1CCC(C)=C[C@H]1c1c(OC(=O)NCC(=O)OCC)cc(CCCCC)cc1OC(=O)NCC(=O)OCC ; C=C(C)[C@@H]1CCC(C)=C[C@H]1c1c(O)cc(CCCCC)cc1OC(=O)NCC(=O)OCC]

A solution of CBD (20.0 g, 63.6 mmol) in 2-MeTHF (192 mL) (or THF) was treated with ethyl 2-isocyanatoacetate (9.99 mL, 89.0 mmol), followed by DBU (13.4 mL, 89.0 mmol). The mixture was stirred at rt for 30-60 min. Upon completion, the mixture was diluted with iPrOAc (100 mL), and a citric acid solution (18.3 g) in 100 mL of water was added. After stirring for 10 min, the organic layer was separate, washed with water (100 mL), brine (100 mL), filtered through a sodium sulfate pad, and concentrated under vacuum to yield a mixture of CBD bis- and mono-glycinate carbamate. The crude was used for the next step without further purification.

### Example 16. Synthesis of CBD Glycine (4)

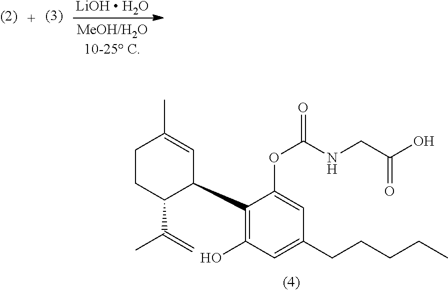 [SMILES: "LiOH · H2O" ; C=C(C)[C@@H]1CCC(C)=C[C@H]1c1c(O)cc(CCCCC)cc1OC(=O)NCC(=O)O]

A solution of CBD bis-glycine carbamate ester (2) and mono-derivative (3) (63.6 mmol) in methanol (283 mL) was treated with a solution of LiOH monohydrate (8.01 g, 191 mmol) in water (287 mL) at 10-25° C. for 20 min. After stirring for 70 min at −23° C., methanol was removed under reduced pressure. The resulting mixture was diluted with water (100 mL) and successively washed with heptane (200 mL), followed by a mixture of iPrOAc (200 mL) and heptane (250 mL). The aqueous layer was separated, and the pH of the aqueous layer was adjusted to 3-4 using citric acid (18.3 g, 95.4 mmol)). Then, the product portion was extracted with MTBE (250 mL). The combined organic layers were washed with water (50 mL), brine (50 mL), filtered through a Na2SO4 pad, and concentrated under vacuum to yield CBD glycine (4). The crude product was used for the next step without further purification.

### Example 17. Synthesis of N,N-dicyclohexylmethylamine CBD glycinate (5a)

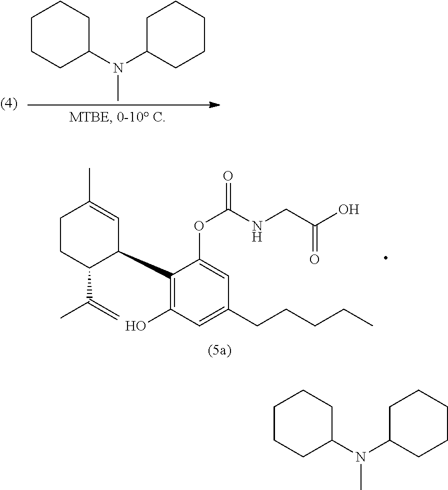 [SMILES: CN(C1CCCCC1)C1CCCCC1 ; C=C(C)[C@@H]1CCC(C)=C[C@H]1c1c(O)cc(CCCCC)cc1OC(=O)NCC(=O)O ; CN(C1CCCCC1)C1CCCCC1]

A solution of CBD glycine (4) (63.6 mmol) in MTBE (303 mL) was treated with N,N-dicyclohexylmethylamine (12.3 mL, 57.2 mmol, 0.90 equiv) at 0-10° C. After stirring for 1-10 h at 0-5° C., the mixture was filtered, washed with MTBE (7 mL) and air-dried to afford N,N-dicyclohexylmethylamine CBD glycinate (5a) (25.12 g) in 65% yield over three steps. HPLC purity, 97.2% (t_{R}=5.24 min); 1H NMR (300 MHz, CD3OD) δ 6.43 (d, J=1.7 Hz, 1H), 6.38 (d, J=1.7 Hz, 1H), 5.19 (s, 1H), 4.55-4.34 (m, 2H), 3.99-3.82 (m, 1H), 3.78 (d, J=17.1 Hz, 1H), 3.62 (d, J=17.1 Hz, 1H), 3.42-3.24 (m, 2H), 2.92-2.75 (m, 1H), 2.71 (s, 3H), 2.46 (t, J=7.7 Hz, 2H), 2.38-2.16 (m, 1H), 2.12-1.84 (m, 9H), 1.81-1.10 (m, 27H), 0.90 (t, J=6.7 Hz, 3H).

### Example 18. Synthesis of Arginine CBD Glycinate (5b)

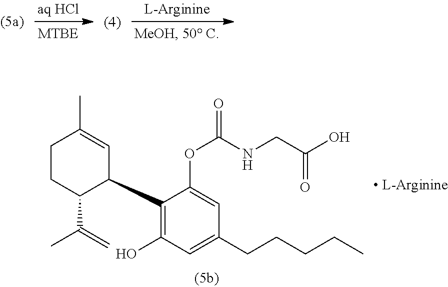 [SMILES: C=C(C)[C@@H]1CCC(C)=C[C@H]1c1c(O)cc(CCCCC)cc1OC(=O)NCC(=O)O]

A solution of N,N-dicyclohexylmethylamine CBD glycinate (5a) (2.02 g, 3.31 mmol) in MTBE (28 mL) was treated with an aqueous HCl solution (13.3 mL, 2 M solution, 26.51 mmol) 5 at rt. The mixture was shaken for 5-10 min, after which the organic layer was separated, washed with water, brine, and then filtered through a Na2SO4 pad. The filtrate was concentrated under vacuum: HPLC purity, 97.3% (tR=5.22 min). The resulting CBD glycine free acid (4) was dissolved in MeOH (20 mL), and L-arginine (548.3 mg, 3.148 mmol, 0.95 equiv) was added. The mixture was stirred for 2 h at 50° C., then stirred at rt for 2 h, filtered, and concentrated under vacuum. The residue was suspended in heptane (20 mL), stirred for 2-10 h at 50° C., filtered, washed with heptane, and dried under vacuum to give arginine CBD glycinate (5b) (1.68 g,) in 86% yield; HPLC purity, 97.1% (t_{R}=5.23 min). The 1H NMR spectrum is identical with that of Example 5.

### Example 19. Synthesis of Carbonate Intermediate (22)

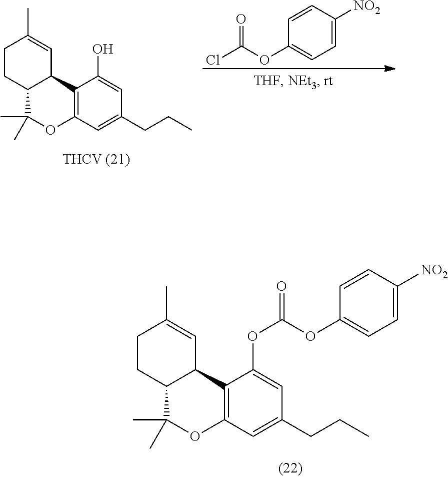 [SMILES: CCCc1cc(O)c2c(c1)OC(C)(C)[C@@H]1CCC(C)=C[C@@H]21 ; O=C(Cl)Oc1ccc([N+](=O)[O-])cc1 ; CCCc1cc(OC(=O)Oc2ccc([N+](=O)[O-])cc2)c2c(c1)OC(C)(C)[C@@H]1CCC(C)=C[C@@H]21]

To a solution of THCV (21) (1102 mg, 3.848 mmol) and 4-nitrophenyl chloroformate (783.3 mg, 3.848 mmol) in 2-MeTHF (16 mL), NEt3 (1.07 mL, 7.70 mmol) was added dropwise. After stirring for an hour, an additional amount of 4-nitrophenyl chloroformate (783.3 mg, 3.848 mmol) was added, and the mixture was stirred at rt for 30 min. Upon completion, the mixture was diluted with iPrOAc (23 mL), washed with 0.1 N HCl (15 mL×3), satd NaHCO3 (15 mL×3), water (15 mL×1), and brine (15 mL), dried (Na2SO4), filtered, and concentrated under vacuum. The crude product was used for the next reaction without further purification.

### Example 20. Synthesis of THCV glycine (23)

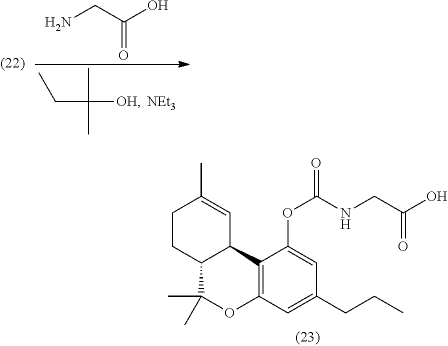

A mixture of carbonate intermediate (22) (1.592 g, 3.526 mmol), obtained in the previous reaction, and glycine (529.4 mg, 7.052 mmol) in t-amyl alcohol (19 mL) was treated with NEt3 (1.97 mL, 14.10 mmol). The mixture was stirred at 70° C. for ˜20 h. After concentrated in vacuo, the residue was purified by column chromatography (SiO2, Eluent; DCM only to 10% MeOH in DCM) to yield the title compound (913 mg, 67%). HPLC purity, >99% (t_{R}=5.62 min).

### Example 21. Synthesis of Arginine THCV Glycinate (24)

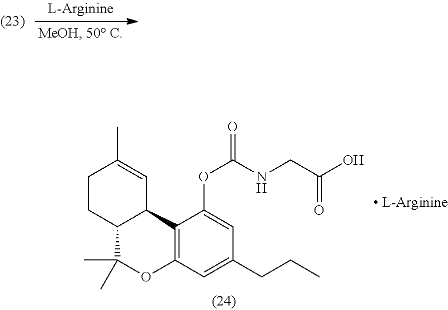 [SMILES: CCCc1cc(OC(=O)NCC(=O)O)c2c(c1)OC(C)(C)[C@@H]1CCC(C)=C[C@@H]21]

A mixture of THCV glycine (23) (913 mg, 2.36 mmol) and L-arginine (246 mg, 1.41 mmol) in MeOH (9.81 g, 12.4 mL, 306 mmol) was stirred at 50° C. for 2 h. After cooling to rt, the mixture was filtered and concentrated in vacuo. The crude product was treated with heptane (20 mL) and heated at 50° C. for 10 h. The mixture was then filtered, washed with heptane, and air-dried to yield the target compound (547 mg, 41%). HPLC purity, 92.4% (t_{R}=5.61 min); 1H NMR (300 MHz, CD3OD) δ 6.50 (d, J=1.7 Hz, 1H), 6.46 (d, J=1.7 Hz, 1H), 6.08 (s, 1H), 3.81 (d, J=17.1 Hz, 1H), 3.67 (d, J=17.1 Hz, 1H), 3.49 (t, J=6.0 Hz, 1H), 3.26-3.05 (m, 3H), 2.46 (t, J=7.6 Hz, 2H), 2.24-2.07 (m, 2H), 1.93 (dd, J=11.9, 1.9 Hz, 1H), 1.89-1.50 (m, 10H), 1.46-1.30 (m, 4H), 1.06 (s, 3H), 0.93 (t, J=7.4 Hz, 3H).

### Example 22. Synthesis of CBG bis-glycine carbamate ester (7) and mono-derivative (8)

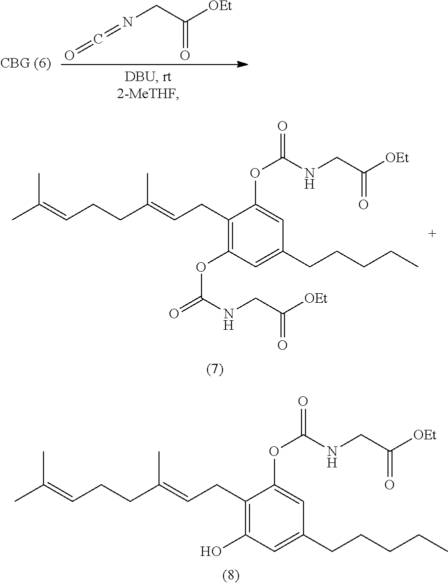

To a solution of CBG (6) (5.00 g, 15.8 mmol) in 2-MeTHF (160 mL), ethyl isocyanatoacetate (3.19 mL, 28.4 mmol) was added at rt, followed by the addition of DBU (4.25 mL, 28.4 mmol, 1.4 equiv). After stirring for 40 min, the mixture was diluted with iPrOAc (150 mL), washed with water (80 mL) and brine (80 mL), dried over Na2SO4, and concentrated under vacuum. The crude product was used in the next step without further purification.

### Example 23. Synthesis of CBG Glycine (9)

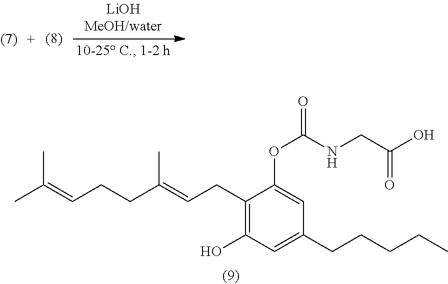 [SMILES: CCCCCc1cc(O)c(C/C=C(\C)CCC=C(C)C)c(OC(=O)NCC(=O)O)c1]

Crude products (7) and (8) (˜9.18 g, 15.8 mmol), obtained from the previous reaction, were dissolved in MeOH (40 mL), and the mixture was treated with a solution of LiOH H2O (1.15 g, 47.9 mmol) in H2O (40 mL) at 15° C. After stirring for 70 min at 15-25° C., the mixture was concentrated in vacuo, diluted with water (170 mL), and successively washed with heptane (80 mL), followed by a mixture of iPrOAc and heptane (80 mL, v/v=1/1). The aqueous layer was separated and acidified with citric acid (4.00 g, 20.8 mmol). The product was extracted with MTBE (100 mL×1 and 50 mL×1). The combined organic layers were washed with water (80 mL), brine, dried over anhydrous Na2SO4, filtered, and concentrated under vacuum to yield the title compound (9) as a yellowish oily liquid with a 61.3% yield (4.087 g, 9.78 mmol). The crude product was used for the next step without further purification. HPLC purity, 95.6% (t_{R}=5.61 min); The 1H NMR spectrum is identical with that of Example 8.

### Example 24. Synthesis of 1,2,3,4-tetrahydro-1-naphthylamine CBG glycinate (10a)

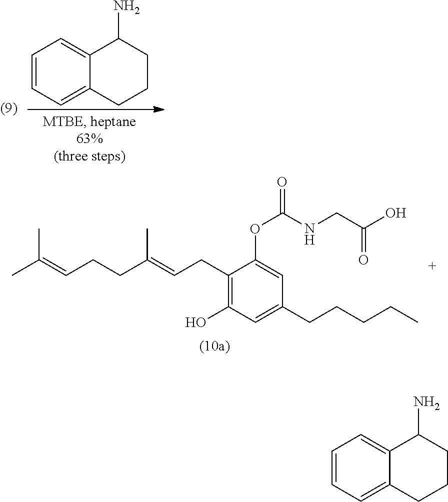 [SMILES: NC1CCCc2ccccc21 ; CCCCCc1cc(O)c(C/C=C(\C)CCC=C(C)C)c(OC(=O)NCC(=O)O)c1 ; NC1CCCc2ccccc21]

To a solution of CBG glycine (9) (4.07 g, 9.75 mmol) in MTBE (32 mL), 1,2,3,4-tetrahydronaphthalen-1-amine (1.05 mL, 7.31 mmol) was added, followed by the addition of heptane (16 mL). After stirring for 16 h, the precipitate was filtered, washed with MTBE (20 mL), and air-dried to yield the title compound (10a) with a 63.0% yield (3.50 g, 6.20 mmol). HPLC purity, 97.9% (t_{R}=5.65 min); 1H NMR (500 MHz, CD3OD) δ 7.38 (dd, J=7.4, 1.7 Hz, 1H), 7.31-7.23 (m, 2H), 7.20 (dd, J=7.4, 1.7 Hz, 1H), 6.49 (d, J=1.6 Hz, 1H), 6.44 (d, J=1.6 Hz, 1H), 5.17 (t, J=7.3 Hz, 1H), 5.07 (t, J=7.3 Hz, 1H), 4.47 (t, J=5.4 Hz, 1H), 3.74 (s, 2H), 3.23 (d, J=7.3 Hz, 2H), 2.89 (dt, J=17.0, 5.7 Hz, 1H), 2.81 (ddd, J=17.0, 8.3, 5.7 Hz, 1H), 2.53-2.39 (m, 2H), 2.23-2.12 (m, 1H), 2.09-1.82 (m, 7H), 1.73 (s, 3H), 1.62 (s, 3H), 1.61-1.54 (m, 5H), 1.41-1.22 (m, 4H), 0.90 (t, J=6.9 Hz, 3H).

### Example 25. Synthesis of Arginine CBG Glycinate (10b)

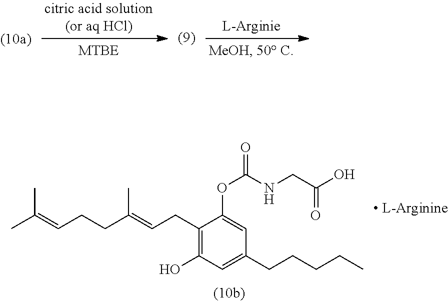 [SMILES: CCCCCc1cc(O)c(C/C=C(\C)CCC=C(C)C)c(OC(=O)NCC(=O)O)c1]

1,2,3,4-Tetrahydro-1-naphthylamine CBG glycinate (10a) (3.50 g, 6.20 mmol) was suspended in MTBE (50 mL) and a 10% citric acid solution (20 mL) was added. The mixture was stirred for several minutes until it became a clear solution. After the layers were separated, the organic layer was washed with water (15 mL×2), brine (15 mL), dried over Na2SO4, and concentrated in vacuo to yield CBG glycine (9) with a 92.2% yield (2.38 g, 5.71 mmol).

To a solution of CBG glycine (9) (2.38 g, 5.71 mmol) in MeOH (22.5 mL), L-arginine (0.945 g, 5.42 mmol) was added, and the mixture was stirred at 50° C. for 5 h. After cooling to rt, the mixture was filtered to remove undissolved particles and concentrated under vacuum. The crude product was suspended in heptane (10 mL) and stirred at 50° C. for 16 h. The precipitate was filtered, washed with a mixture of MTBE and heptane (v/v=1/1), and dried to yield the title compound (10b) as a white solid with a 65.1% yield (2.20 g, 3.72 mmol). HPLC purity, 97.9% (t_{R}=5.60 min); The 1H NMR spectrum is identical with that of Example 9.

### Example 26. Synthesis of carbonate intermediate (16)

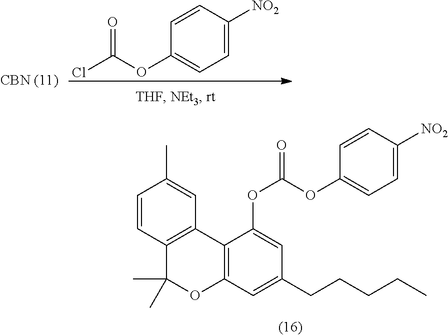 [SMILES: O=C(Cl)Oc1ccc([N+](=O)[O-])cc1 ; CCCCCc1cc(OC(=O)Oc2ccc([N+](=O)[O-])cc2)c2c(c1)OC(C)(C)c1ccc(C)cc1-2]

To a solution of CBN (10.15 g, 32.70 mmol) and 4-nitrophenyl chloroformate (7.249 g, 35.97 mmol) in THF (106 mL), NEt3 (9.11 mL, 65.39 mmol) was added dropwise at 10-25° C. After stirring for 2 h at rt, the mixture was diluted with iPrOAc (106 mL), washed with a citric acid solution (7.4 g, 39 mmol) in water (40 mL), satd NaHCO3 (50 mL), water (50 mL), and brine (50 mL). The organic layer was filtered through a pad of sodium sulfate, washed with iPrOAc (40 mL), and concentrated in vacuo. The crude product was used for the next step without further purification.

### Example 27. Synthesis of CBN γ-aminobutyric acid (GABA) (13)

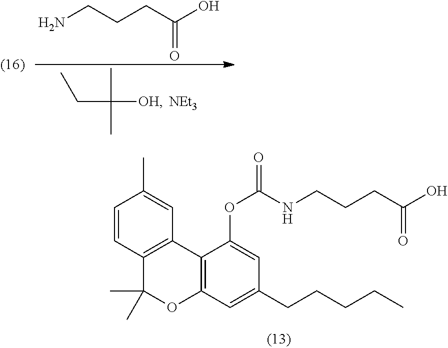

A mixture of carbonate intermediate (16) (15.55 g, 32.70 mmol), obtained from the previous reaction, and γ-aminobutyric acid (GABA) (6.744 g, 65.40 mmol) in t-amyl alcohol (143 mL) was treated with NEt3 (18.20 mL, 130.8 mmol). The mixture was stirred at rt for 20 h. Upon completion, t-amyl alcohol was removed under reduced pressure. The crude product was diluted with MTBE (203 mL), successively washed with a citric acid solution (9.423 g, 49.05 mmol) in water (70 mL), water (70 mL), and brine (70 mL). The organic layer was filtered through a pad of sodium sulfate, washed with MTBE (10 mL), and concentrated in vacuo. The crude product was used for the next step without further purification. 1H NMR (500 MHz, CD3OD) δ 7.98-7.80 (m, 1H), 7.20 (d, J=7.9 Hz, 1H), 7.14-7.06 (m, 1H), 6.68 (d, J=1.7 Hz, 1H), 6.61 (d, J=1.7 Hz, 1H), 3.21 (t, J=6.8 Hz, 2H), 2.58 (t, J=7.3 Hz, 2H), 2.38 (s, 3H), 2.33 (t, J=7.3 Hz, 2H), 1.82 (quint, J=7.2 Hz, 2H), 1.72-1.61 (m, 2H), 1.58 (s, 6H), 1.46-1.28 (m, 4H), 0.93 (t, J=6.9 Hz, 3H).

### Example 28. Synthesis of 1-phenylethylamine CBN γ-aminobutyrate (14a)

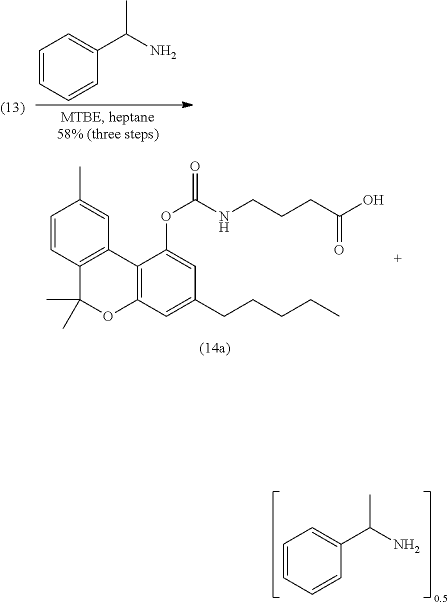

To a solution of CBN γ-aminobutyric acid (13) (14.37 g, 32.70 mmol) in MTBE (100 mL), a solution of 1-phenylethylamine (2.774 g, 22.89 mmol) in MTBE (36 mL) was added, followed by heptane (77 mL). The resulting mixture was stirred at rt for ˜20 h, filtered to collect precipitates, washed with a mixture of MTBE and heptane (80 mL, v/v=1/1), heptane (20 mL), and air-dried to yield the title compound (10.67 g) in 58% yield over three steps. HPLC purity, >99% (t_{R}=6.04 min); 1H NMR (500 MHz, CD3OD) δ 7.86 (s, 1H), 7.51-7.33 (m, 2.5H), 7.17 (d, J=7.9 Hz, 1H), 7.09 (dd, J=7.9, 1.7 Hz, 1H), 6.65 (d, J=1.7 Hz, 1H), 6.58 (d, J=1.7 Hz, 1H), 4.41 (q, J=6.9 Hz, 0.5H), 3.18 (t, J=6.9 Hz, 2H), 2.56 (d, J=7.9 Hz, 2H), 2.36 (s, 3H), 2.27 (t, J=7.5 Hz, 2H), 1.81 (quint, J=7.2 Hz, 2H), 1.66-1.58 (m, 3.5H), 1.56 (s, 6H), 1.43-1.27 (m, 4H), 0.89 (d, J=6.4 Hz, 3H).

### Example 29. Synthesis of arginine CBN γ-aminobutyrate (14b)

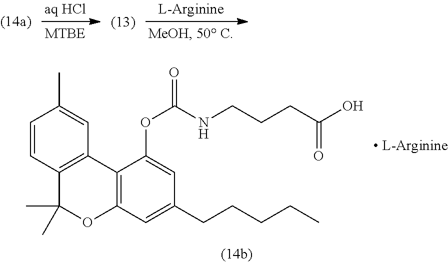 [SMILES: CCCCCc1cc(OC(=O)NCCCC(=O)O)c2c(c1)OC(C)(C)c1ccc(C)cc1-2]

1-Phenylethylamine CBN γ-aminobutyrate (14a) (2.068 g, 3.688 mmol) in MTBE (35 mL) was treated with a solution of aqueous HCl (18.44 mL, 2.0 M) and the mixture was stirred at rt for 10 min. The organic layer was separated, washed with water (10 mL), brine (10 mL), filtered through a pad of sodium sulfate, and washed with MTBE (10 mL). The combined organic layers were concentrated in vacuo, and the residue and L-arginine (583 mg, 3.35 mmol) in MeOH (25 mL) were stirred at 50° C. for 2 h. After cooling to rt, the mixture was filtered to remove any undissolved materials, concentrated under vacuum, and the resulting crude was suspended with heptane (40 mL). The mixture was heated at 50° C. for 2-20 h, cooled to rt, was filtered, washed with heptane, and dried under vacuum to yield the title compound (1.68 g) in 86% yield. HPLC purity, >99% (t_{R}=5.97 min); 1H NMR (500 MHz, CD3OD) δ 7.86 (d, J=1.7 Hz, 1H), 7.17 (d, J=7.9 Hz, 1H), 7.09 (dd, J=8.0, 1.7 Hz, 1H), 6.65 (d, J=1.7 Hz, 1H), 6.58 (d, J=1.7 Hz, 1H), 3.57 (t, J=6.2 Hz, 1H), 3.25-3.12 (m, 4H), 2.56 (d, J=7.6 Hz, 2H), 2.36 (s, 3H), 2.24 (t, J=7.5 Hz, 2H), 1.93-1.78 (m, 4H), 1.77-1.67 (m, 2H), 1.67-1.59 (m, 2H), 1.56 (s, 7H), 1.45-1.27 (m, 4H), 0.92 (t, J=6.9 Hz, 3H).

### Example 30. Synthesis of CBN Taurine (15)

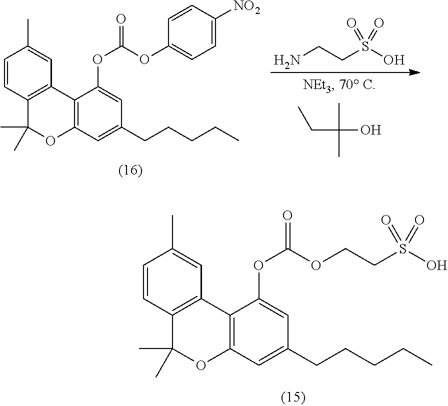

The title compound (15) was prepared using modified conditions. Briefly, a solution of carbonate intermediate (16) (3.367 g, 7.080 mmol) and taurine (1.816 g, 14.51 mmol) in t-amyl alcohol (23.0 mL) was treated with NEt3 (3.95 mL, 28.32 mmol). After stirring for 10 h at 70° C., the mixture was concentrated in vacuo and the residue was diluted with MTBE (40 mL) and water (40 mL). The aq layer was acidified with citric acid (1.572 g, 182 mmol). Then, the organic layer was separated, and the aq layer was back extracted with MTBE (40 mL). The combined organic layers were washed with a citric acid (1.360 g, 7.080 mmol) solution in water, water, and brine. The organic layer was filtered through a Na2SO4 pad, washed with MTBE, and concentrated in vacuo. The residue was purified by column chromatography (SiO2, Eluent; DCM only to 10% MeOH) to yield the title compound (1.1 g, 35%). HPLC purity, 93.3% (t_{R}=4.63 min); 1H NMR (300 MHz, CD3OD) δ 7.81 (d, J=1.7 Hz, 1H), 7.18 (d, J=7.9 Hz, 1H), 7.09 (dd, J=7.9, 1.7 Hz, 1H), 6.66 (d, J=1.7 Hz, 1H), 6.60 (d, J=1.7 Hz, 1H), 3.58 (t, J=6.3 Hz, 2H), 3.01 (t, J=6.3 Hz, 2H), 2.54 (d, J=7.7 Hz, 2H), 2.37 (s, 3H), 1.56 (s, 8H), 1.47-1.22 (m, 4H), 0.91 (t, J=6.4 Hz, 3H).

### Example 31. Synthesis of CBN Glycine (25)

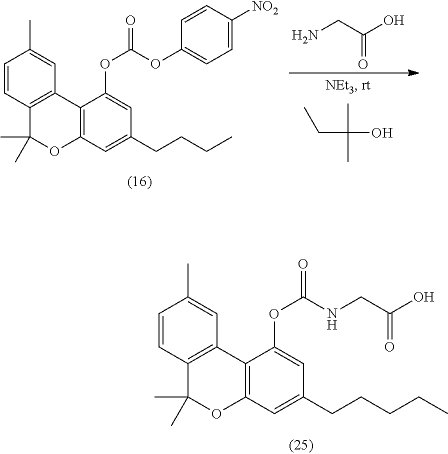

The title compound (1.05 g, 39%) was prepared in a similar manner as that of Example 30 using carbonate intermediate (16) (3.14 g, 6.61 mmol) and glycine (992 mg, 13.2 mmol) in t-amyl alcohol (33.8 mL) and NEt3 (3.69 mL, 26.4 mmol). HPLC purity, 97.8% (t_{R}=5.90 min); 1H NMR (300 MHz, CD3OD) δ 7.91 (s, 1H), 7.21 (d, J=7.9 Hz, 1H), 7.16-7.06 (m, 1H), 6.74-6.61 (m, 2H), 3.77 (s, 2H), 2.59 (t, J=6.8 Hz, 3H), 2.37 (s, 3H), 1.57 (s, 8H), 1.47-1.27 (m, 4H), 0.93 (d, J=7.4 Hz, 3H).

### Example 32. Synthesis of CBD bis-glycine carbamate ester (2) and mono-derivative (3) from carbamoyl imidazolium salt (25)

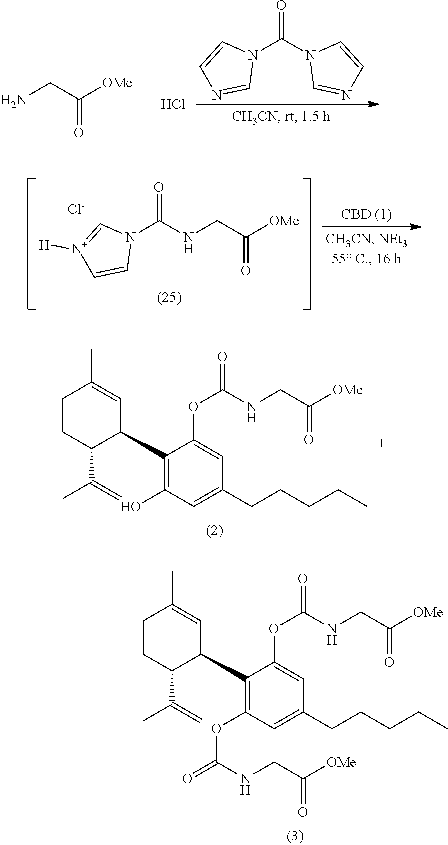

To a solution of carbonyl diimidazole (1.55 g, 9.56 mmol) in CH3CN (10 mL), glycine methyl ester·HCl (1.00 g, 7.96 mmol) was added at rt. The mixture was stirred for 1.5 h and then, filtered. The filtrate was used in the next step without further purification.

CBD (1) (1.50 g, 4.77 mmol) was added to the above solution of carbamoyl imidazolium (25) (1.75 g, 9.54 mmol) in CH3CN, followed by the addition of NEt3 (1.46 mL, 10.5 mmol) at rt. After stirring for 16 h, the reaction mixture was diluted with iPrOAc (30 mL), washed water (20 mL×3), and brine, then dried over anhydrous Na2SO4. The mixture was concentrated under vacuum to yield the title products (2.48 g) as a yellow oil. The crude product was used in the next step without further purification.

### Example 33. Synthesis of CBD Glycine (4) from the Mixture of (2) and (3) from Example 32

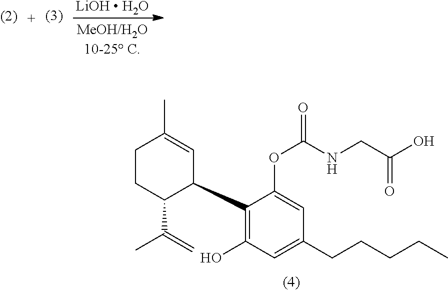 [SMILES: "LiOH · H2O" ; C=C(C)[C@@H]1CCC(C)=C[C@H]1c1c(O)cc(CCCCC)cc1OC(=O)NCC(=O)O]

The title compound (4) was prepared with a 47% yield over three steps (1.13 g, 4.55 mmol) in a similar manner as that of Example 16, using CBD glycine esters (2) and (3) (˜2.50 g, 4.55 mmol) obtained from the previous reaction. The product was for the next step without further purification.

### Example 34. Synthesis of N,N-dicyclohexylmethylamine CBD glycinate (5a)

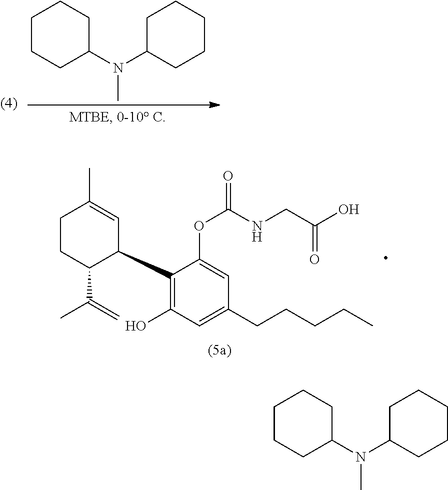 [SMILES: CN(C1CCCCC1)C1CCCCC1 ; C=C(C)[C@@H]1CCC(C)=C[C@H]1c1c(O)cc(CCCCC)cc1OC(=O)NCC(=O)O ; CN(C1CCCCC1)C1CCCCC1]

The title compound (5a) was prepared with a 54% yield (0.900 g, 2.72 mmol) in a similar manner as that of Example 17, using CBD glycine (4) (1.13 g, 2.72 mmol) obtained from the previous reaction and N,N-dicyclohexylmethylamine (0.408 mL, 1.90 mmol).

### Example 35. Synthesis of CBD ethyl γ-aminobutyrate (29) and CBD diethyl γ-aminobutyrate (30) from CBD acyl imidazoles (27) and (28)

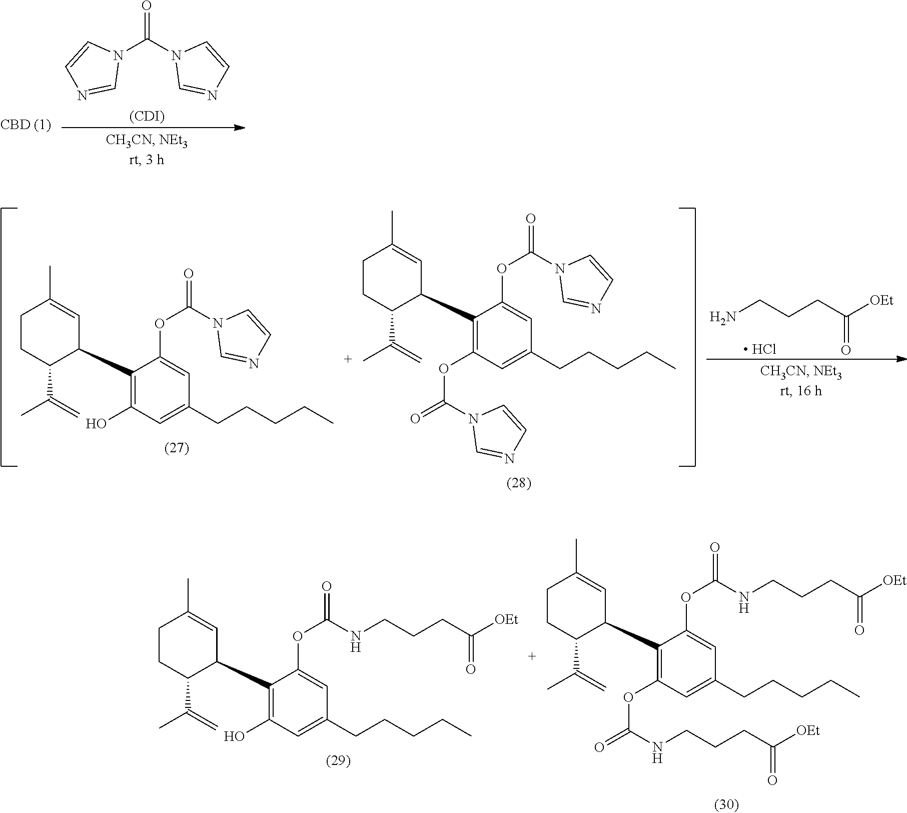

To a solution of CBD (1) (0.500 g, 1.59 mmol) in CH3CN (15 mL), carbonyl diimidazole (0.560 g, 3.50 mmol) and NEt3 (0.480 mL, 3.50 mmol) were added at rt. The mixture was stirred for 3 h. Upon completion, ethyl γ-aminobutyrate (GABA) ester (0.85 g, 5.09 mmol) and NEt3 (1.11 mL, 7.95 mmol) were added sequentially. After stirring for 16 h, the mixture was filtered, concentrated in vacuo. The resulting crude product was diluted with iPrOAc (160 mL), washed with water (80 mL×2), brine (80 mL), dried over Na2SO4, filtered, and concentrated under vacuum. The crude product was purified by column chromatography (SiO2, Eluent: 25% iPrOAc in heptane) to yield the title compounds with a 59% yield over two steps (0.44 g, 0.99 mmol). HPLC purity, 93.9% (tR=6.22 min).

### Example 36. Synthesis of CBD γ-aminobutyric acid (GABA) (31) from the mixture of (29) and (30)

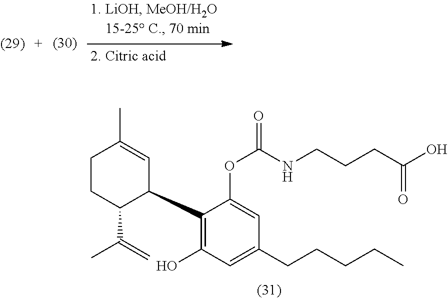 [SMILES: C=C(C)[C@@H]1CCC(C)=C[C@H]1c1c(O)cc(CCCCC)cc1OC(=O)NCCCC(=O)O]

To a solution of (29) and (30) (0.50 g, 0.80 mmol) in MeOH (10 mL) was added a solution of LiOH monohydrate (0.057 g, 2.40 mmol) in water (10 mL) at about 15° C. After stirring for 70 min, the mixture was concentrated in vacuo and diluted with water (20 mL). The aqueous layer was washed with heptane (15 mL), followed by a 1:1 mixture of iPrOAc and heptane (15 mL). The aqueous layer was then acidified with citric acid (0.23 g, 1.2 mmol) and the product was extracted using MTBE (25 mL×2). The combined organic layers were washed with water (20 mL) and brine (20 mL), dried over anhydrous Na2SO4, and concentrated under vacuum to yield the title product (120 mg, 34% yield) as a light-yellow oil after column chromatography (SiO2, Eluent: 5% methanol in DCM). HPLC purity, 89.4% (t_{R}=5.41 min).

### Example 37. Synthesis of Arginine CBD γ-aminobutyrate salt (32) from (31)

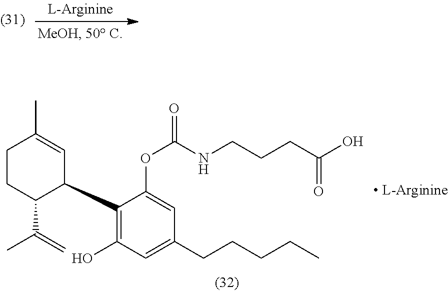 [SMILES: C=C(C)[C@@H]1CCC(C)=C[C@H]1c1c(O)cc(CCCCC)cc1OC(=O)NCCCC(=O)O]

CBD-GABA (31) (100 mg, 0.220 mmol) was dissolved in MeOH (5 mL), and L-arginine (36.5 mg, 0.210 mmol) was added. The mixture was stirred at 50° C. for 10 h. After the solution was filtered through cotton to remove undissolved solid particles, the filtrate was concentrated to yield the title product (97 mg, 70%) as a pale-yellow solid. HPLC purity, 88.9% (t_{R}=5.39 min); 1H NMR (500 MHz, CD3OD) δ 6.44 (d, J=1.7 Hz, 1H), 6.27 (d, J=1.7 Hz, 1H), 5.19 (s, 1H), 4.47 (d, J=2.7 Hz, 1H), 4.43 (d, J=2.7 Hz, 1H), 3.94-3.78 (m, 1H), 3.55 (t, J=6.2 Hz, 1H), 3.27-3.16 (m, 3H), 3.14-3.01 (m, 1H), 2.94-2.77 (m, 1H), 2.46 (d, J=7.6 Hz, 2H), 2.31-2.18 (m, 3H), 2.00-1.93 (m, 1H), 1.90-1.79 (m, 4H), 1.77-1.67 (m, 4H), 1.65-1.61 (m, 6H), 1.57 (quint, J=7.4 Hz, 2H), 1.42-1.20 (m, 4H), 0.90 (t, J=7.0 Hz, 3H).

TABLE 1
Assessment of the aqueous solubility of CBD
and CBG glycine conjugates in salt forms

| | | | | | | Solubility (mg/mL) | |
|---|---|---|---|---|---|---|---|
| | | | Counter | | HPLC | Salt | CBD/CBG |
| Compound | Parent | Promoiety | ion | Mw | purity | derivative | equivalent |
| Example 3 | CBD | glycine | Na | 437.51 | 100% | 4.37 | 3.14 |
| Example 4 | CBD | glycine | L-lysine | 561.72 | 94.1% | 4.06 | 2.27 |
| Example 5 | CBD | glycine | L-arginine | 589.73 | 100% | 12.98 | 6.92 |
| Example 6 | CBD | glycine | meglumine | 610.74 | 100% | 10.03 | 5.16 |
| Example 9 | CBG | glycine | L-arginine | 591.75 | 93.7% | 9.90 | 5.53 |
| Example 10 | CBG | glycine | Na | 439.53 | 100% | 13.30 | 10.00 |

1. A cannabinoid prodrug compound according to Formula I: 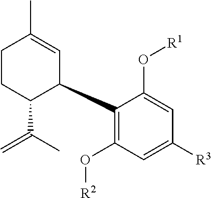
wherein,
R¹ is H, —C(═O)R⁴;
R² is H, —C(═O)R⁴;
R³ is linear alkane;
R⁴ is amino acid, amino acid sugar, or aminosulfonic acid derivative, wherein at least one of R¹ or R² is C(═O)R⁴; or
a pharmaceutically acceptable salt thereof.

2. The compound of claim 1, wherein:
R³ is C₅ linear alkane;
R⁴ is an amino acid-(R⁵), amino acid sugar, or aminosulfonic acid derivative-(R⁵);
R⁵ is H, Na, K, Ca, Mg, amino acid, amino sugar, diethylaminoethanol, or tris base.

3. The compound of claim 1, wherein said amino acid is selected from: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and gamma-aminobutyric acid (GABA).

4. The compound of claim 1, wherein said amino acid sugar is selected: meglumine, glucosamine, galactosamine, sialic acid, and Daunosamine, Mannosamine, Allosamine, Altrosamine, Idosamine, Talosamine, N-Acetyl-D-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, N-Acetyl-6-deoxy-L-altrosamine, N-Acetyl-6-deoxy-D-talosamine.

5. The compound of claim 1, wherein said aminosulfonic acid derivative is a compound having the formula: 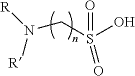
wherein R and R′ are independently selected from the group consisting of hydrogen, alkyl, cyclohexyl, alkoxy, optionally substituted organic groups having one or more hydroxyl groups, optionally substituted organic amide groups, optionally substituted organic sulfonic acids, optionally substituted organic carboxylic acids, optionally substituted organic carboxylic esters, optionally substituted organic amines, and combinations thereof; and
n is 1 to 10.

6. The compound of claim 1, wherein said aminosulfonic acid derivative is —CONHCH₂CH₂SO₃H.

7. A cannabinoid prodrug compound according to Formula II: 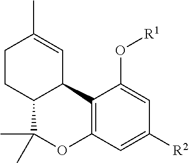
wherein,
R¹ is —C(═O)R³
R² is linear alkane;
R³ is amino acid, amino acid sugar, or aminosulfonic acid derivative; or
a pharmaceutically acceptable salt thereof.

8. The compound of claim 7, wherein:
R² is C₅ linear alkane;
R³ is an amino acid-(R⁴), amino acid sugar, or aminosulfonic acid derivative-(R⁴);
R⁴ is H, Na, K, Ca, Mg, amino acid, amino sugar, diethylaminoethanol, or tris base.

9. The compound of claim 7, wherein said amino acid is selected from: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and GABA.

10. The compound of claim 7, wherein said amino acid sugar is selected from: meglumine, glucosamine, galactosamine, sialic acid, and Daunosamine, Mannosamine, Allosamine, Altrosamine, Idosamine, Talosamine, N-Acetyl-D-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, N-Acetyl-6-deoxy-L-altrosamine, N-Acetyl-6-deoxy-D-talosamine.

11. The compound of claim 7, wherein said aminosulfonic acid derivative is a compound having the formula: 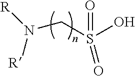
wherein R and R′ are independently selected from the group consisting of hydrogen, alkyl, cyclohexyl, alkoxy, optionally substituted organic groups having one or more hydroxyl groups, optionally substituted organic amide groups, optionally substituted organic sulfonic acids, optionally substituted organic carboxylic acids, optionally substituted organic carboxylic esters, optionally substituted organic amines, and combinations thereof; and
n is 1 to 10.

12. The compound of claim 7, wherein said aminosulfonic acid derivative is —CONHCH₂CH₂SO₃H.

13-18. (canceled)

19. A cannabinoid prodrug compound according to Formula IV: 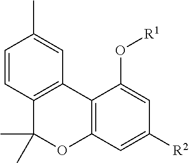
wherein,
R¹ is H, —C(═O)(R³)
R² is linear alkane;
R³ is amino acid, amino acid sugar, or aminosulfonic acid derivative; or
a pharmaceutically acceptable salt thereof.

20. The compound of claim 19, wherein:
R² is C₅ linear alkane;
R³ is an amino acid-(R⁴) or amino acid sugar, or aminosulfonic acid derivative-(R⁴);
R⁴ is H, Na, K, Ca, Mg, amino acid, amino sugar, diethylaminoethanol, or tris base.

21. The compound of claim 19, wherein said amino acid is selected from: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and GABA.

22. The compound of claim 19, wherein said amino acid sugar is selected from: meglumine, glucosamine, galactosamine, sialic acid, and Daunosamine, Mannosamine, Allosamine, Altrosamine, Idosamine, Talosamine, N-Acetyl-D-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, N-Acetyl-6-deoxy-L-altrosamine, N-Acetyl-6-deoxy-D-talosamine.

23. The compound of claim 19, wherein said aminosulfonic acid derivative is a compound having the formula: 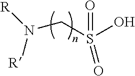
wherein R and R′ are independently selected from the group consisting of hydrogen, alkyl, cyclohexyl, alkoxy, optionally substituted organic groups having one or more hydroxyl groups, optionally substituted organic amide groups, optionally substituted organic sulfonic acids, optionally substituted organic carboxylic acids, optionally substituted organic carboxylic esters, optionally substituted organic amines, and combinations thereof; and
n is 1 to 10.

24. The compound of claim 19, wherein said aminosulfonic acid derivative is —CONHCH₂CH₂SO₃H.

25-69. (canceled)

70. A pharmaceutical composition comprising the compound of claim 1, and a pharmaceutically acceptable carrier.

71. A pharmaceutical composition comprising the compound of claim 7, and a pharmaceutically acceptable carrier.

72. A pharmaceutical composition comprising the compound of claim 19, and a pharmaceutically acceptable carrier.
